(19) United States
(12) Patent Application Publication
(10) Pub. No.: US 2026/0366018 A1
(43) Pub. Date: Nov. 19, 2026

(54) LincRNA-p21 AND USE THEREOF

(71) Applicant: CHINA MEDICAL UNIVERSITY, Taichung City (TW)

(72) Inventors: Wei-Chien Huang, Taichung City (TW); Yu-Hao He, Taipei City (TW); Ya-Ling Wei, Taichung City (TW); Chun-Hsu Yao, Yunlin County (TW); Der-Yang Cho, Taichung City (TW)

(21) Appl. No.: 19/100,227

(22) PCT Filed: Jul. 27, 2023
(86) PCT No.: PCT/CN2023/109496

§ 371 Date: Jan. 31, 2025

Related U.S. Application Data
(60) Provisional application No. 63/370,111, filed on Aug. 2, 2022.

(51) Int. Cl. C12N 15/113; A61K 9/50; A61K 31/555; A61K 31/704; A61K 33/243; A61K 47/68; A61K 47/69; A61P 35/00; C07K 16/28

(52) U.S. Cl. CPC C12N 15/113; A61K 9/5068; A61K 31/555; A61K 31/704; A61K 33/243; A61K 47/6849; A61K 47/6901; A61P 35/00; C07K 16/2833; C12N 2310/14

(57) ABSTRACT

Provided is a composition for treating cancer comprising a DDB2 inhibitor and a chemotherapeutic agent, in which the DDB2 inhibitor comprises three RNA fragments from lincRNA-p21. In addition, the DDB2 inhibitor enhances the chemosensitivity of the cancer to the chemotherapeutic agent for treating cancers that do not respond to chemotherapy.

### FIELD OF THE INVENTION

The present invention relates to a composition for treating cancer, wherein the composition comprises three RNA fragments from lincRNA-p21 and a chemotherapeutic agent. In particular, the three RNA fragments from lincRNA-p21 having DDB2-targeting activity improve the cellular sensitivity of various cancers to the chemotherapeutic agent.

### DESCRIPTION OF PRIOR ART

In response to treatments with chemotherapeutic agents, DNA damage response (DDR) occurs and activates p53 to transcriptionally regulate gene expressions, which decide the cell fate toward senescence, cell cycle progression, cell apoptosis, or DNA repair. High DNA repair activity through nucleotide excision repair (NER), base excision repair (BER), homologous recombination (HR), or non-homologous end joining (NHEJ) in cancer cells contributes to the development of drug resistance to chemotherapy. Targeting various DDR or DNA repair components has been viewed as promising therapeutic strategies in cancer. The exquisite sensitivity of BRCA1/2-mutant tumors to the inhibition of poly (ADP-ribose) polymerase (PARP) leads to the success of clinical treatments with PARP inhibitors. Therefore, synthetic lethality by co-targeting various DNA repair/DDR pathways has provided a paradigm for the development of novel and potential clinical strategies.

Among DNA repair mechanisms, NER plays a critical role in the removal of cisplatin- or doxorubicin-induced DNA damage. Upon the stimulation with chemotherapy, damaged DNA-binding protein 2 (DDB2) is upregulated by activated p53 to act as the first protein recognizing the damaged DNA. DNA-bound DDB2 is then poly-ubiquitinated and proteasomal degraded to handover the damage DNA sites to the second recognition protein XPC for the further recruitment of other DNA repair proteins involved in NER. DDB2 expression is induced by DNA damaging agents, including doxorubicin, and confers to chemoresistance. Mutation or deficiency of DDB2 reduces the recognition of damaged DNA as well as the recruitment of NER-associated proteins, leading to failure of DNA repair. Moreover, PARP1 is also reported to promote NER efficacy by interacting and stabilizing DDB2 protein expression. Suppression of DDB2 increases the cellular sensitivity of triple-negative breast cancer to PARP inhibitors by destabilizing Rad51, indicating an additional role of DDB2 in modulating HR. In addition to DNA repair, DDB2 activity occurs at several stages of tumor progression including cancer cell proliferation, survival, epithelial to mesenchymal transition, migration and invasion, and cancer stem cell formation. Therefore, targeting DDB2 is a potential strategy to increase the chemosensitivity and anti-cancer activity of PARP inhibitors. However, there is no DDB2 inhibitor or modulator available for cancer therapy.

Although the nucleic acid therapy can also be applied in the treatment of cancer, the issues of RNA stability, delivery, and structure are still a problem, and RNA therapy still trails behind other therapies in terms of strategies for treating cancer.

Since most of the long non-coding RNAs (lncRNAs) are at least 200nt in length, it is quite difficult to use lncRNA as a therapeutic strategy for RNA therapy. Therefore, lncRNAs have received little attention and application in clinical practice, and most lncRNAs are considered to be disease markers rather than therapeutics.

### DETAILED DESCRIPTION OF THE INVENTION

The present invention has demonstrated the inverse correlation between lincRNA-p21 and DDB2 in different subtypes of mutp53-expressing breast cancer cell lines and clinical specimens. The increased lincRNA-p21 is shown to enhance the poly-ubiquitination and proteasomal degradation of DDB2 via acting as a scaffold of the Cul-4/DDB1/DDB2 E3-ligase complex. The downregulation of DDB2 by lincRNA-p21 is demonstrated to repress DNA repair. More importantly, three essential elements of lincRNA-p21 comprising 5′-CUUGUGUCCCCUUCCCACAG-3′ (671nt-690nt; #3) (SEQ ID NO: 1); 5′-CAGGGAACCCCUUCAAUCCC-3′ (875nt-894nt; #4) (SEQ ID NO: 2); and 5′-UGGGAGCCCCCUUCCUAAAA-3′ (2, 158nt-2, 177nt; #9) (SEQ ID NO: 3) for direct interaction and suppression of DDB2 protein are identified in different binding assays. The structural binding capacity is also calculated to reveal the possibility of short lincRNA-p21 elements affecting the stability of DDB2 and DNA repair. Co-treatments with short lincRNA-p21 elements or the exosome containing the short lincRNA-p21 elements are found to enhance chemotherapy-induced cytotoxicity in cancer cells.

The short lincRNA-p21 elements using exosome as the delivery system functions as lncRNA-based DDB2 inhibitors show the potential to enhance chemosensitivity and may benefit patients with breast or other cancer types who fail to respond to chemotherapy.

As used herein, the term “a” or “an” are employed to describe elements and components of the present invention. This is done merely for convenience and to give a general sense of the present invention. This description should be read to include one or at least one and the singular also includes the plural unless it is obvious that it is meant otherwise.

The term “or” as used herein may mean “and/or.”

The present invention provides a nucleic acid molecule comprising a sequence of long intergenic non-coding RNA-p21 (lincRNA-p21), wherein the sequence of lincRNA-p21 is selected from the group consisting of

(SEQ ID NO: 1)
CUUGUGUCCCCUUCCCACAG,
(SEQ ID NO: 2)
CAGGGAACCCCUUCAAUCCC
and
(SEQ ID NO: 3)
UGGGAGCCCCCUUCCUAAAA.

The present invention also provides a composition comprising a sequence of lincRNA-p21, wherein the sequence of lincRNA-p21 is selected from the group consisting of SEQ ID NO: 1, SEQ ID NO: 2 and SEQ ID NO: 3.

In addition, the present invention further provides a method for treating cancer, comprising administering a composition into a subject suffering from cancer, wherein the composition comprises a sequence of lincRNA-p21 and a chemotherapeutic agent, wherein the sequence of lincRNA-p21 is selected from the group consisting of SEQ ID NO: 1, SEQ ID NO: 2 and SEQ ID NO: 3.

The present invention provides a use of a composition for preparing a drug for treating cancer, wherein the composition comprises a sequence of lincRNA-p21 and a chemotherapeutic agent, wherein the sequence of lincRNA-p21 is selected from the group consisting of SEQ ID NO: 1, SEQ ID NO: 2 and SEQ ID NO: 3.

The present invention also provides a composition for use in the treatment of cancer, wherein the composition comprises a sequence of lincRNA-p21 and a chemotherapeutic agent, wherein the sequence of lincRNA-p21 is selected from the group consisting of SEQ ID NO: 1, SEQ ID NO: 2 and SEQ ID NO: 3.

The term “subject” as used herein refers to an animal, especially a mammal. In a preferred embodiment, the subject is a human.

Damaged DNA binding protein 2 (DDB2) is an important protein that recognizes DNA damage to initiate DNA repair and make cancer cells resistant to chemotherapeutic agents. In the present invention, the three short sequences from the lincRNA-p21 interfere with the DNA damage repair pathway. Furthermore, the sequence of the lincRNA-p21 is able to inhibit DNA repair induced by DDB2 to enhance the anti-cancer effect of the chemotherapeutic agents. Therefore, the sequence of the lincRNA-p21 inhibits the expression of DDB2 to reverse or reduce cancer cell resistance to the chemotherapeutic agent and/or enhance the sensitivity of the cancer cells to the chemotherapeutic agent. In one embodiment, the sequence of lincRNA-p21 enhances the sensitivity of cancer to the chemotherapeutic agent by inhibiting the expression of DDB2. Thus, DDB2 can be identified as a therapeutic target of cancer. In one embodiment, the cancer comprises the cancer with the high expression of DDB2. In the present invention, the cancer with high expression of DDB2 means that the expression of DDB2 in the tumor tissue has 1.5-fold expression more than that in the normal tissue. In another embodiment, the cancer has poor response or drug-resistance for the chemotherapeutic agent. In a preferred embodiment, the cancer with the high expression of DDB2 has poor response or drug-resistance for the chemotherapeutic agent.

In some aspects, the chemotherapeutic agent is an anticancer drug. In one embodiment, the cancer is the cancer with drug resistance. Therefore, the cancer has drug-resistance to the chemotherapeutic agent. In the present invention, it provides a method of decreasing drug-resistance to a chemotherapeutic agent for treating cancer, comprising administering a composition into a subject suffering from cancer with drug resistance, wherein the composition comprises a sequence of lincRNA-p21 and the chemotherapeutic agent, wherein the sequence of lincRNA-p21 is selected from the group consisting of SEQ ID NO: 1, SEQ ID NO: 2 and SEQ ID NO: 3. The sequence of lincRNA-p21 can reverse or reduce cancer cell resistance to the chemotherapeutic agent and/or enhance the sensitivity of the cancer cells to the chemotherapeutic agent.

The term “treating” encompasses, but is not limited to, reducing, inhibiting or limiting the growth of cancer cells, reducing, inhibiting or limiting metastasis of the cancer cells or invasiveness of the cancer cells or metastasis or reducing, inhibiting or limiting one or more symptoms of cancer or metastasis thereof.

In one embodiment, the cancer comprises breast cancer, liver cancer, cholangiocarcinoma, lung cancer, colon cancer, head and neck squamous cell carcinoma, stomach adenocarcinoma, and esophageal carcinoma. In a preferred embodiment, the cancer comprises breast cancer and liver cancer. In a preferred embodiment, the cancer comprises breast cancer.

In another embodiment, the cancer cells of the cancer have mutant p53. In a preferred embodiment, the cancer cells of the breast cancer have mutant p53. In a more preferred embodiment, the cancer cells of the breast cancer are estrogen receptor (ER)-positive and have mutant p53.

In one embodiment, the breast cancer has poor response or drug-resistance to the chemotherapeutic agent.

As used herein, the chemotherapeutic agent is compounds which can inhibit the growth of cancer cells or tumors. It is understood that one or more chemotherapeutic agents can be used in any of the methods set forth herein. For example, two or more chemotherapeutic agents, three or more chemotherapeutic agents, four or more chemotherapeutic agents, etc. can be used in the methods provided herein. Exemplary chemotherapeutic agents include, without limitation, anti-cancer compounds such as cyclophosphamide, doxorubicin, 5-fluorouracil, docetaxel, paclitaxel, methotrexate, epirubicin, cisplatin, carboplatin, vinorelbine, capecitabine, gemcitabine, mitoxantrone, isabepilone, eribulin, carmustine, a nitrogen mustard, a sulfur mustard, a platin tetranitrate, vinblastine, etoposide, camptothecin, a topoisomerase inhibitors, as well as derivatives, or one or more combinations thereof. In one embodiment, the chemotherapeutic agent comprises carboplatin, cisplatin or doxorubicin.

In some aspects, the sequence of the lincRNA-p21 is effective in enhancing the therapeutic effects of the chemotherapeutic agent. As used herein, the term “enhancing the therapeutic effects” includes any of a number of subjective or objective factors indicating a beneficial response or improvement of the condition being treated as discussed herein. For example, enhancing the therapeutic effects of the chemotherapeutic agent includes reversing or reducing cancer cell resistance and/or enhancing the sensitivity of a drug-resistant cancer to the therapy of the chemotherapeutic agent. Also, for example, enhancing the therapeutic effects of the chemotherapeutic agent includes altering drug-resistant cancer cells so that the cells are not resistant to the chemotherapeutic agent. Also, for example, enhancing the therapeutic effects of the chemotherapeutic agent includes additively or synergistically improving or increasing the activity of the chemotherapeutic agent.

In the present invention, the composition comprises one or more sequences of lincRNA-p21 and one or more chemotherapeutic agents. In one embodiment, the composition further comprises a pharmaceutically acceptable carrier. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. In other embodiments, the pharmaceutically acceptable carrier comprises a liposome, a nanoparticle, an exosome, a micelle, a polymeric matrix or a gel matrix. In the present invention, the sequence of lincRNA-p21 is contained in, or is in a complex with, the liposome, nanoparticle, exosome, micelle, polymeric matrix or gel matrix. In one embodiment, the pharmaceutically acceptable carrier comprises a liposome or an exosome.

In the present invention, the sequence of lincRNA-p21 is able to be loaded into the exosome. In another embodiment, the pharmaceutically acceptable carrier comprises an exosome, wherein the sequence of lincRNA-p21 is contained in the exosome. The exosome containing the sequence of lincRNA-p21 is prepared for treating cancer. In addition, the exosome can bind to an anti-human leukocyte antigen G (HLAG) antibody to form an anti-HLAG exosome. Because the HLAG is highly expressed in a wide variety of cancers, the use of the anti-HLAG antibody is to increase the delivery efficiency of the exosome containing the sequence of lincRNA-p21 and the chemotherapeutic agent to cancer cells. In one embodiment, the composition further comprises a target molecule for binding to a biomarker on cancer cells. In a preferred embodiment, the target molecule comprises an anti-HLAG antibody. Therefore, the anti-HLAG antibody can bind to the sequence of lincRNA-p21 or the exosome to form a therapeutic complex for use in the treatment of cancer.

In the methods provided herein, the sequence of lincRNA-p21 can be administered to the subject prior to, simultaneously, or after administration of the chemotherapeutic agent. In addition, the compositions of the present invention can be administered by any of a variety of routes including: by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intravesicular, intrapulmonary, intracranial, intraventricular, intraspinal, dermal, subdermal, intra-articular, placement within cavities of the body, nasal inhalation, pulmonary inhalation, impression into skin, and electroporation. Administration can be systemic or local. Pharmaceutical compositions can be delivered locally to the area in need of treatment, for example by topical application or local injection. Multiple administrations and/or dosages can also be used.

In the present invention, the subject is administered a therapeutically effective amount of the composition comprising the sequence of lincRNA-p21 and the chemotherapeutic agent. The term “therapeutically effective amount” is defined as any amount necessary to produce a desired physiologic response. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like.

Dosage amounts of the sequence of lincRNA-p21 or the chemotherapeutic agent are typically in the range of from about 0.0001, 0.001 or 0.01 mg/kg/day to about 1000 mg/kg/day, but can be higher or lower, depending upon, among other factors, the activity of the composition, its bioavailability, the mode of administration, and various factors discussed above. The dosage amount and interval can be adjusted individually to provide local and/or systemic concentrations of the exosomes that are sufficient to maintain therapeutic or prophylactic effects. For example, the composition can be administered once per week, several times per week (e.g., every other day), once per day, or multiple times per day, depending upon, among other things, the mode of administration, the specific indication being treated, and the judgment of the prescribing physician. Skilled artisans will be able to optimize effective local dosages without undue experimentation. In one embodiment, the therapeutically effective amount of the composition ranges from 0.01 to 100 mg/kg weight. In a preferred embodiment, the therapeutically effective amount of the composition ranges from 0.1 to 50 mg/kg weight. In a more preferred embodiment, the therapeutically effective amount of the composition ranges from 1 to 10 mg/kg weight.

In thus, chemotherapy resistance is a major problem in the clinical treatment of various cancers. Among these, DNA repair induced by the DDB2 protein is one of the main reasons why cancer cells are insensitive to chemotherapy. The present invention mainly finds that lincRNA-p21 can directly bind to DDB2 and cause its degradation, so it can be used as the first inhibitor of DDB2, which can improve clinical chemotherapy drugs such as carboplatin, cisplatin, and doxorubicin.

More importantly, using different experimental modes to find three basic short sequences necessary for the binding of lincRNA-p21 and DDB2 protein, computer prediction and calculation show that these three short lincRNA-p21 sequences can be bound to the region of DDB2, which interacts with DDB1 proteins. The molecular interface between them stabilizes the formation of the Cul-4/DDB1/DDB2 complex. Without the need for full-length lincRNA-p21, these three sequences can still bind directly to the DDB2 protein, promote proteolysis of DDB2, and increase the sensitivity of cancer cells to chemotherapeutic drugs. Since the length of lincRNA-p21 is more than 3,000 nucleotides, if the full-length lincRNA-p21 is used as an RNA therapy strategy, the synthesis, delivery and stability of the product are quite difficult. Another important breakthrough of this present invention is to prove that only three short lincRNA-p21 sequences of about 20 nucleotides each can be directly combined with DDB2, and the mechanism of action can be analyzed by molecular biology and molecular simulation. It can achieve the functions of DDB2 protein degradation, DNA repair inhibition and chemosensitivity enhancement.

More importantly, the present invention uses the exosome to coat three short sequences of lincRNA-p21 (exoLinc-p21s) with the chemotherapy drug doxorubicin as a drug delivery model and demonstrated that exoLinc-p21s can enhance the toxicity and growth inhibition of doxorubicin in cancer cells. In addition, the anti-HLAG exosome is further used as an RNA delivery system to identify cancer cells, and the anti-HLAG antibody loaded on the exosome can improve the delivery efficiency of exoLinc-p21s and chemotherapeutic drugs to cancer cells. The results prove that the anti-HLAG exosome can not only achieve the effect of promoting exoLinc-p21s to cause DDB2 proteolysis and tumor cell toxicity, but also increase the efficiency of delivery to tumors to increase the sensitivity to chemotherapy drugs.

In conclusion, three short sequences of lincRNA-p21 essential for binding with DDB2 (Linc-p21s) are identified and developed as the first-in-class DDB2 inhibitor with the advantages in low cost of synthesis, high stability and delivery efficacy than full-length lincRNA-p21. Linc-p21s have been shown to stabilize the molecular interface between DDB2 and DDB1 proteins in molecular simulation analysis, and are demonstrated to directly bind to DDB2 protein for its proteasomal degradation. By using the exosome expressing cancer-targeting αHLAG antibody as the delivery system, the exosomal Linc-p21s (exoLinc-p21s) packaged with chemotherapeutic agents is proved to enhance the cytotoxicity and growth inhibitory effects of doxorubicin on cancer cells in cell line and animal models. As the first-in-class DDB2 inhibitor, exoLinc-p21s has the potential to be developed as a novel RNA-based chemosensitizer to benefit patients with various cancer types.

### BRIEF DESCRIPTION OF THE DRAWINGS

FIGS. 1A-1C show that lincRNA-p21 expression is negatively correlated with stage, tumor size, and ERα status. FIGS. 1A and 1B show that lincRNA-p21 expression quantified by in situ hybridization (ISH) assay is higher in early (stage IIA, n=12; stage IIB, n=12) than in late (stage IIIA, n=8; stage IIIB, n=8) human breast cancer tumors (FIG. 1A) and negatively correlated with tumor size (FIG. 1B).

FIG. 1C shows that lincRNA-p21 expression quantified by ISH assay is higher in ERα-negative (n=27) than in ERα-positive (n=13) human breast cancer tumors. The arrows indicate the signal of lincRNA-p21 expression as calculated by the average number of dots per nucleus. Welch two-sample t-test: * p<0.05, ** p<0.01, *** p<0.001.

FIGS. 2A-2J show that higher lincRNA-p21 expression is expressed in early-stage ERα-negative breast cancer with smaller tumor size, and contributes to chemosensitivity in breast cancer. FIG. 2A shows an illustration of the treatment timeline in the Tet-On-LincRNA-p21 tumor-xenograft mouse model (arrow: the starting point of 0.2 mg/mL tetracycline administration) (Top). FIG. 2A also shows that the growth rate of T47D human breast xenograft tumors is inhibited by tetracycline-induced lincRNA-p21 expression (Bottom). Data are representative of three independent experiments in each group and are presented as mean±SD. * p<0.05; ** p<0.01; *** p<0.001 versus the control group, Student's t-test. FIGS. 2B, 2C and 2D show that the ex vivo induction of lincRNA-p21 expression by carboplatin (50 μM) is negatively correlated with disease stage (FIG. 2B) and tumor size (n=61) (FIG. 2C) and was higher in ERα-negative (n=14) than in ERα-positive (n=47) human primary breast cancer tissues (FIG. 2D). Welch two-sample t-test: * p<0.05, ** p<0.01, *** p<0.001. FIG. 2E shows induction of lincRNA-p21 expression is negatively correlated with the IC₅₀ of breast cancer cell lines in response to chemotherapy. FIGS. 2F and 2G show that ectopic expression (FIG. 2F) and silencing (FIG. 2G) of lincRNA-p21 alters carboplatin-induced apoptotic death of T-47D cancer cells in the fluorescence-activated cell sorting (FACS) assay. FIG. 2H shows that silencing of lincRNA-p21 reduces carboplatin-induced the expression of apoptotic markers in MDA-MB-231 cancer cells. FIGS. 2I and 2J show that silencing of lincRNA-p21 by two independent shRNAs suppresses the chemosensitizing effect of tamoxifen (FIG. 2I) and ERα silencing (FIG. 2J) in BT-474 cancer cells, as evidenced by the induction of apoptotic death measured by the FACS assay. Data in (FIGS. 2F, 2G, 2I, and 2J) are representative of at least three experiments and are shown as mean±SD. * p<0.05; ** p<0.01; *** p<0.001 versus control group, Student's t-test.

FIGS. 3A and 3B show that lincRNA-p21 is an intermediator in the ERα-associated chemoresistance. FIGS. 3A and 3B show the raw data of FIGS. 2I and 2J in the FACS assay. Data are representative of at least three experiments and are shown as mean±SD. * p<0.05; ** p<0.01; *** p<0.001 versus control group, Student's t-test.

FIGS. 4A-4K show that lincRNA-p21 reduces DNA repair and is negatively correlated with DDB2 expression. FIG. 4A shows that lincRNA-p21 silencing reduces cisplatin (50 μM)-induced cisplatin-DNA adduct (Pt-(GpG) purine dimmer) formation in MDA-MB-231 cancer cells in a time-dependent manner in an immunofluorescence assay. Images from the immunofluorescence assay are quantified by using ImageJ analysis. FIG. 4B shows that the network of protein-coding genes associated with Erα-positive expression is analyzed using STRING and Cytoscape 3.8.0. FIG. 4C shows that DDB2 expression analyzed in the GSE18908 dataset is higher in Erα-positive than in Erα-negative human breast cancer tissues. Welch two-sample t-test: * p<0.05, ** p<0.01, *** p<0.001. FIG. 4D shows that higher DDB2 expression shows worse overall survival (OS, n=187) in Erα-positive breast cancer patients receiving neoadjuvant chemotherapy in Kaplan-Meier survival analysis. FIGS. 4E and 4F show that ex vivo induction of DDB2 mRNA expression by carboplatin (50 μM) is higher in Erα-positive (n=47) than in Erα-negative (n=14) human primary breast cancer tissues (FIG. 4E), Welch two-sample t-test: * p<0.05, ** p<0.01, *** p<0.001, and positively correlated with tumor size (n=61) (FIG. 4F). FIG. 4G shows chemotherapy response (CR: complete response (100% deceased), PR: partial response (>=50%, <100% decreased), SD: stable disease (<50% decreased), PD: partial disease (0% decreased)) in neoadjuvant patients with induction of lincRNA-p21 and DDB2 levels. FIGS. 4H and 4I show that chemotherapy-induced dynamic protein expression (FIG. 4H) and the chromatin-binding activity (FIG. 4I) of DDB2 are higher in Erα-positive than in Erα-negative breast cancer cell lines. FIG. 4J shows that knockdown of DDB2 by two independent shRNAs enhances the expression of carboplatin-induced apoptotic markers in a dose-(left) and a time-(right) dependent manner in BT-474 cancer cells in the western blot analysis. FIG. 4K shows that knockdown of DDB2 enhances carboplatin-induced apoptotic death of T-47D cancer cells in the FASC assay. Data are representative of at least three experiments and are shown as mean±SD. * p<0.05; ** p<0.01; *** p<0.001 versus control group, Student's t-test.

FIGS. 5A-5E show that DDB2 contributes to DNA repair function for chemoresistance. FIG. 5A shows the ranking of ERα-associated gene expressions in human breast tumors in the Kyoto Encyclopedia of Genes and Genomes (KEGG) pathway analysis. FIG. 5B show that higher DDB2 expression is associated with worse overall survival (OS, n=76) in ERα-positive/mutp53 breast cancer patients receiving neoadjuvant chemotherapy compare to ERα-negative/mutp53 breast cancer patients in Kaplan-Meier survival analysis. FIG. 5C shows that the box plot of DDB2 expression in each cancer type is analyzed from the pan-cancer database, GEPIA.

FIG. 5D shows that the efficacy of DNA repair in T-47D and MDA-MB-231 cancer cells in response to cisplatin (50 μM) is examined in a time-dependent manner in an immunofluorescence assay using an anti-cisplatin modified DNA antibody. Images from the immunofluorescence assay are quantified by using ImageJ analysis. FIG. 5E shows that DDB2 expression is induced by chemotherapy in ERα-positive but not ERα-negative breast cancer cells.

FIGS. 6A and 6B show that lincRNA-p21 may target DDB2 and interfere with its nuclear co-localization for chemosensitization. FIGS. 6A and 6B show that carboplatin (50 μM)-induced nuclear translocated protein accumulation (FIG. 6A) and doxorubicin (0.5 μM)-enriched DDB2 levels in the triton-resistant (chromatin-bound) lysates (FIG. 6B) in ERα-positive but not-negative breast cancer cell lines.

FIGS. 7A-7K show that lincRNA-p21 downregulates DDB2 expression by enhancing the formation of the Cul-4/DDB1/DDB2 E3-ligase complex. FIG. 7A shows that carboplatin (50 μM) and doxorubicin (0.5 μM) induce lincRNA-p21 expression in a time-dependent manner in ERα-negative MDA-MB-231 cancer cells (top and right) but not in ERα-positive T-47D cancer cells (top and left). In contrast, DDB2 mRNA levels are induced by these chemotherapeutic agents in the ERα-positive breast cancer cell line (lower and left). FIG. 7B shows that nuclear-translocation of lincRNA-p21 is induced by carboplatin in a time-dependent manner in ERα-negative MDA-MB-231 cancer cells (right) but not in ERα-positive T-47D cancer cells (left). FIG. 7C shows that ectopic expression (left) and silencing (right) of lincRNA-p21 do not affect DDB2 mRNA levels in T-47D and MDA-MB-231 cancer cells in qRT-PCR analysis. FIG. 7D shows that DDB2 mRNA levels are not altered by lincRNA-p21 induction in T-47D #Tet-On-LincRNA-p21 cancer cells. FIG. 7E shows that treatment with the proteasome inhibitor MG132 (10 μM) increases DDB2 expression in a time-dependent manner. FIG. 7F shows the raw data of FIG. 8D in the western blot assay. FIG. 7G shows doxorubicin (0.5 μM)-induced an in vivo association of lincRNA-p21 with DDB2 (left), DDB1 and Cul-4 (right), in MDA-MB-231 cancer cells by RNA-IP assay. FIG. 7H shows that in vitro treatment with RNase A reduces the carboplatin-induced complex formation of DDB2 with DDB1 and Cul-4 in the co-IP assay. FIG. 7I shows that knockdown of lincRNA-p21 reduces DDB2 protein levels in the anti-Cul-4 and anti-DDB1 immunocomplex in response to carboplatin (50 μM) in the presence of MG132. FIG. 7J shows that DDB2 from carboplatin-treated T-47D cancer cell lysates is pulled down in vitro by different segments of biotinylated lincRNA-p21. The dot plot reveals an equal input of the biotinylated RNAs. FIG. 7K shows carboplatin (50 μM)-induced the in vivo association of DDB1 and Cul-4 with lincRNA-p21 at a specific region followed by RNase A digestion manner in T-47D cancer cells by RNA-IP analysis. Data in FIGS. 7A, 7C, 7D, 7G, and 7K are representative of three experiments and are shown as mean±SD. * p<0.05; ** p<0.01; *** p<0.001 versus control group, Student's t-test.

FIGS. 8A-8N show that lincRNA-p21 enhances DDB2 protein ubiquitination and degradation by acting as a scaffold of the Cul-4/DDB1/DDB2 complex. FIG. 8A shows that ectopic expression of lincRNA-p21 decreases DDB2 protein levels. FIG. 8B shows the knockdown of lincRNA-p21 increases DDB2 protein levels. FIG. 8C shows that DDB2 protein expression is suppressed by lincRNA-p21 induction in the stable cancer cell clone of the T-47D #Tet-On system. FIG. 8D shows that ectopic expression of lincRNA-p21 reduces the stability of DDB2 protein in the presence of CHX (25 μM). The level of DDB2 protein examined in western blot analysis is quantified using ImageJ and normalized with α-Tubulin. FIG. 8E shows that pretreatment with MG132 (10 μM) prevents the lincRNA-p21-induced DDB2 downregulation. FIG. 8F shows that ectopic expression of lincRNA-p21 increases the poly-ubiquitination of DDB2 in MG132-treated T-47D cancer cells. FIGS. 8G and 8H show carboplatin (50 μM)-induced the in vivo association of DDB2 (FIG. 8G), DDB1, and Cul-4 (FIG. 8H) with lincRNA-p21 in ERα-negative MDA-MB-231 cancer cells but not in ERα-positive T-47D cancer cells in the RNA-IP assay. FIG. 8I shows that knockdown of lincRNA-p21 reduces the carboplatin-induced complex formation of DDB2 with DDB1 and Cul-4 in the co-IP assay. FIG. 8J shows that DDB2, DDB1, and Cul-4 from carboplatin-treated T-47D cancer cell lysates are pulled down in vitro by the biotinylated lincRNA-p21, but not by HOTAIR or α-Tubulin mRNA. The dot plot reveals an equal input of the biotinylated RNAs. hnRNP-K is used as a positive control for lincRNA-p21 interacting protein. FIG. 8K shows an illustration of the deleted fragments of biotinylated lincRNA-p21 for RNA pull-down assay and the primer sets at different regions for RNA-IP. FIG. 8L shows that DDB2 from carboplatin-treated T-47D cancer cell lysates is pulled down in vitro by different deleted fragments of biotinylated lincRNA-p21. The dot plot reveals an equal input of the biotinylated RNAs. FIG. 8M shows carboplatin (50 μM)-induced the in vivo association of DDB2 with lincRNA-p21 at a specific region followed by RNase A digestion manner in T-47D cancer cells by RNA-IP analysis. Data in FIGS. 8G, 8H, and 8M are representative of at least three experiments and are shown as mean±SD. * p<0.05; ** p<0.01; *** p<0.001 versus control group, Student's t-test. FIG. 8N shows an illustration of the predicted secondary structure of lincRNA-p21 (ViennaRNA web server), and the putative DDB2 binding elements.

FIGS. 9A-9I show that three short lincRNA-p21 elements, #3, #4, and #9, bind to the DDB2 protein in vitro. FIGS. 9A-9D show that single (FIGS. 9A and 9B) or complex (FIGS. 9C and 9D) deletion mutants of biotinylated lincRNA-p21 are subjected to pull-down assay with DDB2 protein from carboplatin-treated T-47D cancer cell lysates in vitro. The dot plot reveals an equal input of the biotinylated RNAs. FIG. 9E shows the dose-dependent binding activity of short lincRNA-p21 elements, #3, #4, and #9, to recombinant DDB2 protein in surface plasmon resonance (SPR) assay. FIG. 9F shows that the Ct value of the gradient concentration of pure short lincRNA-p21 elements is used as the standard curve. FIG. 9G shows that the transfection efficiency of short lincRNA-p21 elements, #3, #4, and #9 into T-47D cancer cells is comparable at different concentrations. FIG. 9H shows that combinatorial co-treatment with all three short lincRNA-p21 elements dramatically enhances cytotoxicity in the presence or absence of carboplatin, cisplatin, and doxorubicin. Data are representative of three experiments and are shown as mean±SD. * p<0.05; ** p<0.01; *** p<0.001 versus control group, Student's t-test. FIG. 9I shows that the inhibitory effect is reversed by treatment with MG132.

FIGS. 10A-10C show the effects of short lincRNA-p21 elements, #3, #4, and #9 on chemotherapy-induced cytotoxicity in breast cancer cells. FIG. 10A shows that treatment with three short lincRNA-p21 elements alone increases cytotoxicity only in the presence of carboplatin but has no effect on cisplatin and doxorubicin. Data are representative of three experiments and are shown as mean±SD. * p<0.05; ** p<0.01; *** p<0.001 versus control group, Student's t-test. FIG. 10B shows that combined co-treatment with all three short lincRNA-p21 elements can suppress the DDB2 protein expression. FIG. 10C shows the raw data of FIG. 9I.

FIGS. 11A-11E show 3D structural modeling of short lincRNA-p21 elements in complex with the N-terminal α-helix of DDB2 by computational molecular docking. FIG. 11A shows three 3D structures of short lincRNA-p21 elements are calculated and predicted by six databases from RNAComposer: CentroidFold, CONTRAfold, IPknot, RNAfold, RNAstructure, and ContextFold. FIG. 11B shows that two thousand poses are calculated by using the ZDOCK docking program in BIOVIA Discovery Studio and six angles per pose are generated. The marked dots indicate the potential poses between two macromolecules and the arrowed dot is the selected short lincRNA-p21 elements with the highest potential interaction with DDB2. FIG. 11C shows the clusters of potential poses around the N-terminal α-helix of DDB2. FIG. 11D shows that models of three 3D structures of short lincRNA-p21 elements coil around the DDB2 α-helix, which is responsible for interaction with DDB1. FIG. 11E shows the interaction sites between short lincRNA-p21 elements and DDB2 in 3D structure.

FIGS. 12A-12H show the positions of the short lincRNA-p21 elements in complex with DDB2 by computational molecular docking. FIGS. 12A-12E show that docking results from other databases and the selected poses are shown as marked dots, indicating the potential poses between two macromolecules. FIGS. 12F-12H show the most potential conformation prediction between short lincRNA-p21 elements and DDB2 in other databases. In FIG. 12F, the aquamarine indicates 5 database (pose 22), and the pink indicates contextFold (pose 27). In FIG. 12G, the aquamarine indicates 4 database (pose 19), the pink indicates contextFold (pose 2), and the yellow indicates RNAstructure (pose 1). In FIG. 12H, the aquamarine indicates 4 database (pose 8), the pink indicates contextFold (pose 40), and the yellow indicates RNAstructure (pose 8).

FIGS. 13A-13H show that the exosome-packaged short lincRNA-p21 elements, #3, #4, and #9 (exoLinc-p21s), enhance the chemotherapy-induced cytotoxicity in breast cancer cells. FIG. 13A shows the exosome particle imaged by TEM. FIGS. 13B-13F show that all three short lincRNA-p21 elements (#3+ #4+ #9) are packaged by exosome (exoLinc-p21s) as an exosome-based therapy to demonstrate DNA repair inhibition function (FIG. 13B), DDB2 protein inhibition function (FIG. 13C), growth inhibition function (FIG. 13D), cytotoxicity enhancement effects (FIG. 13E), and tumor size inhibition in xenograft mouse model (FIG. 13F). FIGS. 13G and 13H show cytotoxicity effect (FIG. 13G) and DDB2 protein inhibition function (FIG. 13H) in exoLinc-p21s with and without an anti-HLAG coating. Data in FIGS. 13D, 13E, and 13G are representative of at least three experiments and are shown as mean±SD. * p<0.05; ** p<0.01; *** p<0.001 versus control group, Student's t-test.

FIGS. 14A and 14B show the delivery efficiency of exosomes with and without an anti-HLAG. FIG. 14A shows the colony area and average colony size in the growth inhibition function of exoLinc-p21s. FIG. 14B shows the delivery efficiency of exosomes with and without an anti-HLAG in a time-dependent manner by immunofluorescence assay.

### EXAMPLES

The present invention may be implemented in many different forms and should not be construed as limited to the examples set forth herein. The described examples are not limited to the scope of the present invention as described in the claims.

### Material and Methods

### Clinical Specimen

A total of 61 residual breast cancer tissue specimens were collected, after obtaining informed consent, from patients undergoing surgery for different breast cancer subtypes in Chung Shan Medical University Hospital, Taichung, Taiwan. Collection of samples included non-selected subtypes and the samples were used according to a protocol approved by the Institutional Review Board of Chung Shan Medical University Hospital, Taichung, Taiwan (CS2-18150). Tissues were homogenised and cultured with or without carboplatin for 5 days. Following treatment, total RNA and protein lysates were prepared with TRIzol™ Reagent (Thermo Fisher Scientific Inc., Waltham, MA, USA).

### Tissue Microarray and In Situ Hybridization

Breast cancer tissue microarrays were purchased from SuperBioChips Laboratories (Seoul, Korea) and used to detect lincRNA-p21 for in situ hybridization experiments. Tissue microarray specimens included different breast cancer subtypes from 40 patients. The RNAscope lincRNA-p21 (TP53COR1) probe for using in the in situ hybridization assay was designed and purchased from Advanced Cell Diagnostics, Inc. (Newark, CA, USA). The present invention used the RNAscope 2.5 HD Detection Kit-BROWN according to the manufacturer's protocol to screen for lincRNA-p21 signaling in breast cancer tissues. Signals for lincRNA-p21 expression were quantified with Fiji ImageJ and normalized with nuclei to calculate the areas and percentages of probe numbers.

### Cell Culture

Breast cancer cell lines, MCF7 (RRID: CVCL_0031), T-47D (RRID: CVCL_0553), BT-474 (RRID: CVCL_0179), SK-BR-3 (RRID: CVCL_0033), MDA-MB-468 (RRID: CVCL_0419), and MDA-MB-231 (RRID: CVCL_0062) as well as liver cancer cell lines, HepG2 (RRID: CVCL_0027) were cultured in Dulbecco's Modified Eagle Medium: Nutrient Mixture F-12 (DMEM/F12, HyClone™, Thermo Fisher Scientific Inc., Waltham, MA, USA), supplemented with 10% fetal bovine serum (FBS, Gibco, Thermo Fisher Scientific Inc., Waltham, MA, USA) and HyClone™ Penicillin-Streptomycin Solution. All cell lines were purchased from the American Type Culture Collection (ATCC) and incubated at 37 °C in a humidified incubator containing 5% CO₂ and examined non-contamination from mycoplasma by MycoAlert™ Mycoplasma Detection Kit (LT07-318, Thermo Fisher Scientific Inc., Waltham, MA, USA).

### Inhibitors and Reagents

Carboplatin (41575-94-4), (Z)-4-hydroxy Tamoxifen (68047-06-3), cycloheximide (CHX, 66-81-9), (S)-MG132 (133407-82-6), and Tetracycline (hydrochloride, 64-75-5) were purchased from Cayman Chemical (Michigan, USA). Cisplatin (cis-diammineplatinum (II), P4394) and doxorubicin hydrochloride (Sigma-Aldrich, D1515) were purchased from Merck KGaA (Darmstadt, Germany). Clarity™ western enhanced chemiluminescence (ECL) substrate was purchased from Bio-Rad Laboratories, Inc., (Hercules, CA, USA).

### Antibodies

Antibodies against DDB2 (#5416, RRID: AB_10694497), Ac-p53 (K382, #2525S, RRID: AB_330083), p-ERα (Ser118, #2511), HA-Tag (#3724, RRID: AB_1549585), PARP (#9542, RRID: AB_2160739), and Histone H3 (#9715, RRID: AB_331563) were purchased from Cell Signaling Technology, Inc., (Beverly, MA, USA). Antibodies against DDB1 (sc-25367, RRID: AB_639050) Cul-4 (sc-377188), hnRNP-K (sc-28380), p53 (sc-126, RRID: AB_628082), and ERα (sc-8002, RRID: AB_627558) were purchased from Santa Cruz Biotechnology, Inc., (CA, USA). Antibodies against ubiquitin (P4D1-A11), p21WAF1 (Calbiochem, OP64, RRID: AB_2335868), α-Tubulin (T5168, RRID: AB_477579), and β-Actin (A2228, RRID: AB_476697) were purchased from Merck KGaA (Darmstadt, Germany). Antibody against Caspase3 (Imgenex IMG-144A, RRID: AB_316677) was purchased from Novus Biologicals, LLC., (Centennial, CO, USA). Antibody against p-Histone H2AX (Ser139, AF2288, RRID: AB_2114989) was purchased from R&D Systems Inc., (Minneapolis, MN, USA).

### Western Blot Analysis

Total protein lysate concentration was determined using the Bradford protein assay (Bio-Rad Laboratories, Inc., Hercules, CA, USA), whereby 30 μg of protein lysate was heated at 95 °C in sample buffer for 5 minutes. Denatured proteins were separated in SDS-PAGE with a running buffer and transferred to PVDF membranes (0.45 μM, Millipore, Merck KGaA, Darmstadt, Germany) or NC membranes (0.22 μM, Amersh™, GE Healthcare Life Science, Pittsburgh, PA, USA) with transfer buffer. The transferred membrane was blocked with 5% milk or BSA in TBST buffer and stained with the indicated primary antibodies at 4 °C overnight, followed by incubation with HRP-conjugated secondary antibodies. ECL signaling was detected using a ChemiDoc™ Touch Imaging System (Bio-Rad).

### RNA Extraction and RT-PCR

After the indicated treatments, cells were washed three times with ice-cold PBS and lysed with TRIzol™ Reagent (Thermo Fisher Scientific Inc., Waltham, MA, USA). Total RNA was isolated by adding 0.2 mL of chloroform per 1 mL of TRIzol™ Reagent, followed by centrifugation at 12,000 g for 15 minutes to separate the aqueous, interphase and organic phases. Next, RNA from the aqueous phase was precipitated by mixing with 0.25-0.5 mL of isopropanol, followed by centrifugation at 12,000 g for 15 minutes. After removal of supernatant, the gel-like pellet was washed twice with 1 mL of 75% ethanol, air-dried, and then dissolved in DEPC-treated water. The reverse transcription polymerase chain reaction (RT-PCR) was performed with 1 μg total RNA, Invitrogen™ M-MLV Reverse Transcriptase (Thermo Fisher Scientific Inc., Waltham, MA, USA), Random Hexamer, dNTP, 5× M-MLV buffer, and DTT.

### Quantitative Real-Time PCR

For qRT-PCR, KAPA SYBR FAST qPCR Master Mix (2×) Kit (Kapa Biosystems, Wilmington, MA, USA) was used to detect the expression of target genes with specific primers. The threshold cycle or Ct value was analyzed using the LightCycler 480 Real-Time PCR System (Roche Molecular Systems, Inc., Pleasanton, CA, USA) or Applied Biosystems™ QuantStudio™ 5 Real-Time PCR System (Thermo Fisher Scientific Inc., Waltham, MA, USA). ddCt was calculated with normalisation to housekeeping genes as the reference.

### RNA Immunoprecipitation (RNA-IP) Assay Protocol

Cells were fixated with 1% formaldehyde and neutralized using 1M glycine, washed twice with ice-cold PBS, then scraped and vortexed with lysis buffer (50 mM HEPES pH7.5, 150 mM NaCl, 1% Triton X-100, 0.1% SDS, 1 mM DTT, cOmplete™ Protease Inhibitor Cocktail (1 tablet contains protease inhibitors sufficient for a 10 mL cell extract) (Roche Molecular Systems, Inc., Pleasanton, CA, USA), and 200 U/mL RNaseOUT™ (Thermo Fisher Scientific Inc., Waltham, MA, USA). After undergoing 3 freeze-thaw cycles on ice, the lysates were centrifuged at 14,000 rpm for 30 minutes and the supernatants were collected for immunoprecipitation. Agarose protein A/G was pre-blocked for an hour then mixed and incubated overnight with antibody in NT2 buffer (50 mM Tris-HCl pH7.5, 150 mM NaCl, 1 mM MgCl₂, and 0.5% NP-40). They were then washed 3 times with NT2 buffer and incubated with lysate NT2 buffer (1 mM DTT, 200 U/mL RNaseOUT™, and 20 mM EDTA) and rotated overnight. The immunocomplexes were washed 3 times and then reverse-crosslinked in 100 μL of NT2 buffer at 70 °C for 5 hours. Finally, samples were incubated with 0.25 mg/mL Sigma-Aldrich Proteinase K (Merck KGaA, Darmstadt, Germany) at 55 °C for 30 minutes and lysed with TRIzol™ Reagent (Thermo Fisher Scientific Inc., Waltham, MA, USA) for RNA extraction and qRT-PCR.

### Preparation of In Vitro Transcription of Biotinylated RNA

To examine the in vitro interactions of RNA with target proteins, including DDB1, DDB2, Cul-4, and hnRNP-K, biotinylated RNAs prepared with Invitrogen™ T7 RNA Polymerase (Thermo Fisher Scientific Inc., Waltham, MA, USA) and Biotin RNA Labeling Mix (Roche Molecular Systems, Inc., Pleasanton, CA, USA) were used in the biotin pull-down assay. Biotinylated lincRNA-p21, HOTAIR, and tubulin RNA were generated by using their DNA templates synthesized in PCR with T7-containing primer sets.

### Biotin Pull-Down Assay Protocol

For the in vitro pull-down assay, 3 μg of biotin-labelled RNA was heated to 90 °C for 2 minutes, and restructured in structure buffer (10 mM Tris-HCl to pH7.0, 0.1 M KCl, and 10 mM MgCl₂) at room temperature for 20 minutes. Cells (2×10⁷) were treated with or without chemotherapy treatments, then resuspended in nuclear isolation buffer (1.28 M sucrose, 40 mM Tris-HCl pH7.5, 20 mM MgCl₂, and 4% Triton X-100). The nuclei pellets were hybridized to the folded DNA or RNA in RIP buffer (150 mM KCl, 25 mM Tris-HCl pH7.4, 0.5 mM DTT, 0.5% NP-40, 1 mM PMSF, and cOmplete™ Protease Inhibitor Cocktail (1 tablet contains protease inhibitors sufficient for a 10 mL cell extract) (Roche Molecular Systems, Inc., Pleasanton, CA, USA)) for an hour, then RNA-protein complexes were pulled down using Novagen streptavidin agarose beads (Novagen Corporation, San Diego, CA, USA) and analyzed by western blot.

### Fractionation of Nuclear and Cytoplasmic RNA

Cells were washed twice with TD buffer (137 mM NaCl, 5 mM KCl, 0.7 mM Na₂ HPO₄, and 25 mM Tris-HCl pH7.4), before they were scraped off with TD buffer followed by centrifugation at maximum speed for 30 seconds at room temperature. The pellet was washed with 200 μL of TD buffer, resuspended with 100 μL of Vanadyl Ribonucleoside Complex buffer (20 mM VRC; S1402S, New England BioLabs Inc., Ipswich, MA, USA, 10 mM Tris-HCl, 0.14 M NaCl, 1.5 mM MgCl₂, 1 mM DTT, and 0.5% NP-40 in TD buffer pH8.6), vortexed for 10 seconds, then incubated on ice for 5 minutes. The cells were then vortexed again and centrifuged at maximum speed for 30 seconds, before the supernatant was transferred to a new eppendorf centrifuge tube and lysed with TRIzol™ Reagent (Thermo Fisher Scientific Inc., Waltham, MA, USA) to isolate cytoplasmic RNA. For isolating nuclear RNA, the pellets were washed with 200 μL of 0.5% NP-40/TD buffer, resuspended with 100 μL of 0.5% NP-40/TD buffer, then lysed with Invitrogen TRIzol™ Reagent.

### Triton Extraction Assay

After treatment with carboplatin, cells were lysed with Triton extraction buffer (100 mM NaCl, 300 mM sucrose, 3 mM MgCl₂, 10 mM PIPES pH6.8, 1 mM EGTA pH6.8, 0.2% Triton X-100, with freshly added 1 mM NaVO₄, 1 mM PMSF, 10 mM NaF, and 1 ng/mL aprotinin) at 4 °C for 30 minutes. Supernatant was collected as a Triton-extractable fraction (chromatin-free protein), while the pellet was collected as the Triton-resistant fraction (chromatin-bound protein), which was washed twice with Triton extraction buffer and then further lysed with NETN buffer (20 mM Tris-HCl pH8.0, 150 mM NaCl, 1 mM EDTA, 0.5% NP-40, with freshly added 1 mM NaVO₄, 1 mM PMSF, 10 mM NaF, and 1 ng/mL aprotinin). Both fractions were used to examine the free and DNA-bound forms of DDB2, respectively.

### Cell Viability Assay Protocol

The MTT assay was used to detect cell viability. Cells (5×10³) grown in 96-well plates were treated with different concentrations of the indicated chemotherapy treatments for 48 or 72 hours. The culture medium was then changed to serum-free medium containing 5× Sigma-Aldrich MTT solution (Merck KGaA, Darmstadt, Germany) and incubated for 2.5 hours. The cells were then lysed with DMSO and the optical density (OD) at 570 nm was detected by an ELISA reader.

### Quantification of Cisplatin Adducts in Nuclear DNA by Immunocytological Assay

The effect of lincRNA-p21 and DDB2 on DNA repair was investigated by detecting cisplatin adducts in immunofluorescence staining with anti-cisplatin modified DNA antibody. After treatment with cisplatin (50 μM) for the indicated times to induce DNA damage, cells were fixed with 4% paraformaldehyde and covered with 1% Triton X-100 at room temperature for 5-7 minutes after PBS washed. The cells were then blocked with 1% BSA in PBS at room temperature for an hour and were stained with anti-cisplatin modified DNA antibody (Abcam, plc., Cambridge, England) at room temperature for an hour in darkness. This was followed by staining with a second antibody, goat anti-rat IgG H&L (Abcam, plc., Cambridge, England), at room temperature for another hours in darkness. Finally, the cells were mounted with DAPI mounting medium (Thermo Fisher Scientific Inc., Waltham, MA, USA) and staining was observed with a fluorescent microscope (Leica DMIL LED, Leica Microsystems, Wetzlar, Germany). Signaling of cisplatin adducts was quantitated using ImageJ software and was normalized against nuclei DAPI signals.

### Macromolecular Docking

The schematic of DDB2 (4E54) was found in the protein data bank (PDB). Short lincRNA-p21 #3, #4, and #9 3D structure were predicted and created by the RNAcomposer database and further used for ZDOCK docking between DDB2 through BIOVIA Discovery Studio software (RRID: SCR_015651). The docking results were presented in 3D structure by BIOVIA Discovery Studio and PyMoL software (RRID: SCR_000305).

### Xenograft Mouse Model

The breast tumor xenograft mouse model was used to validate the growth effect of lincRNA-p21 through the Tet-On system, and the synergism between exoLinc-p21s and exoDox. T-47D breast cancer cells were injected into the mammary fat pad of five-week-old female BALB/c nude mice that had been implanted subcutaneously with 60-day release pellets containing 0.7-mg 17β-estradiol (Innovative Research of America) 3 days prior to subcutaneous inoculation. After 1 month of tumor growth in mice, the suppressive effect of exoScramble, exoLinc-p21s, and the combination treatments with exoDox on tumor growth was determined. During the treatment period, the activity of the mice was monitored and the survival curve between the four treatment groups of treatments was calculated, and the tumor diameters were measured serially with calipers, and tumor volume was calculated using the formula: volume=length×width²/2.

### Statistical Analysis

The difference between two categorical variables was analyzed using the Student's t-test or Welch's two-sample t-test, while differences among more than two category variables were analyzed by one-way ANOVA. The results were presented as the mean±SD, n≥3. The p-value was calculated with the two-tailed test, and a statistically significant difference was defined as p<0.05. All statistical analysis was performed using SigmaPlot 10.0, GraphPad Prism 8, or SPSS 21 software.

### Results

LincRNA-p21 suppresses ERα/DDB2-associated DNA repair and chemoresistance.

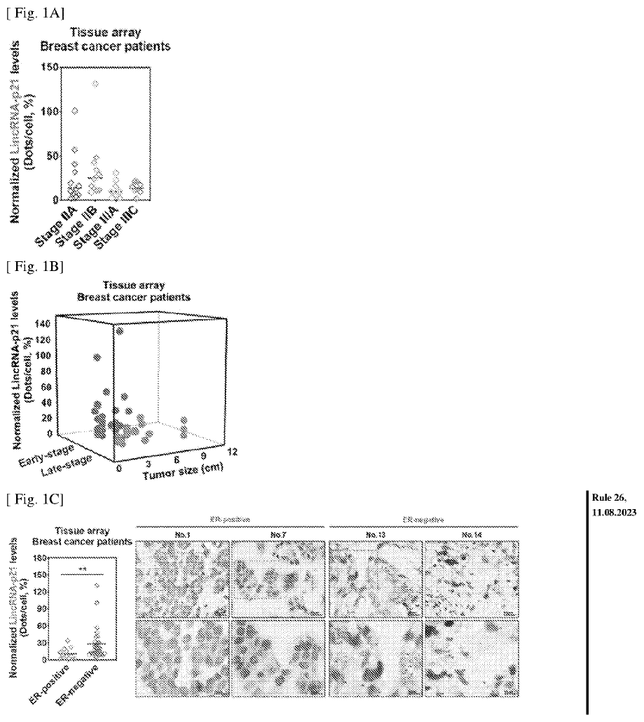

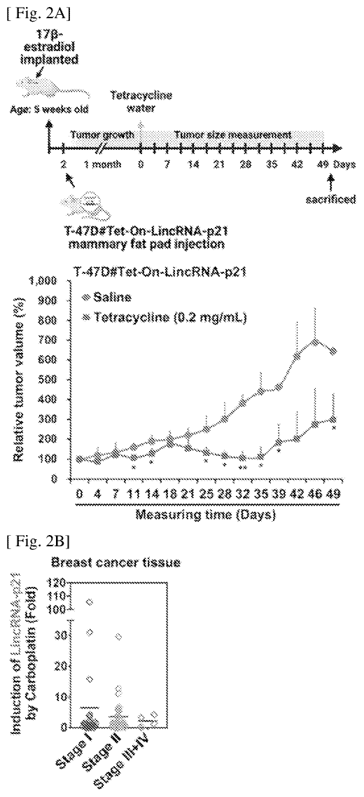

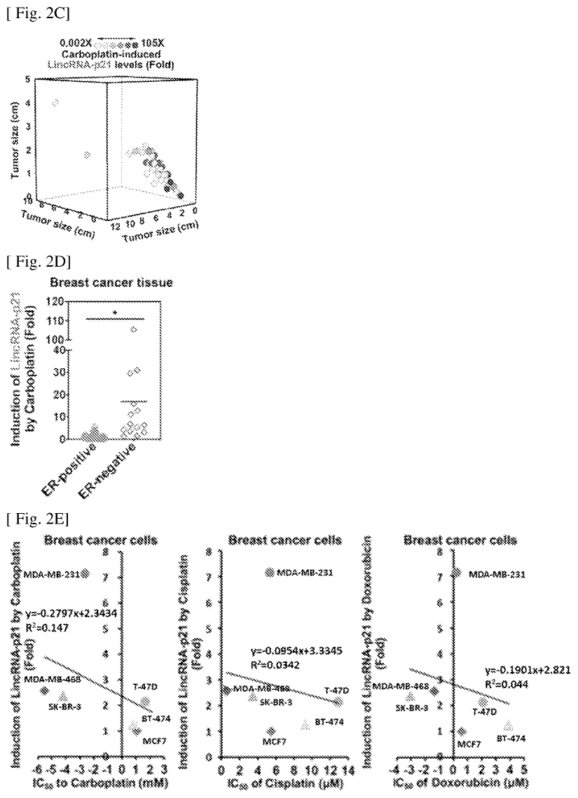

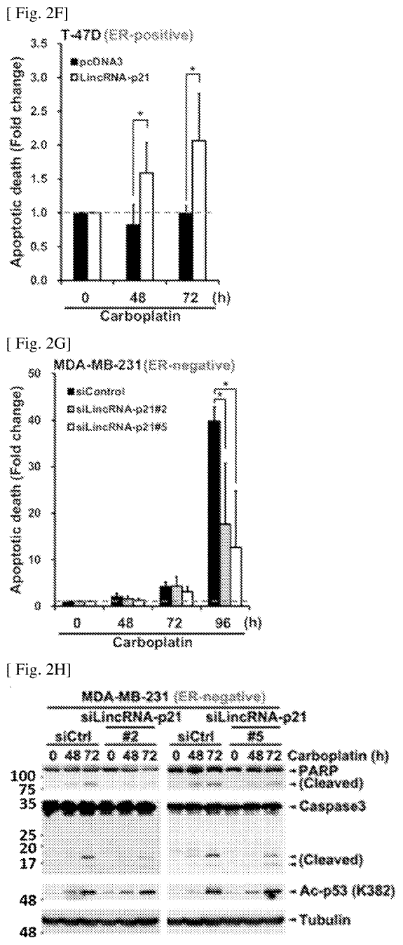

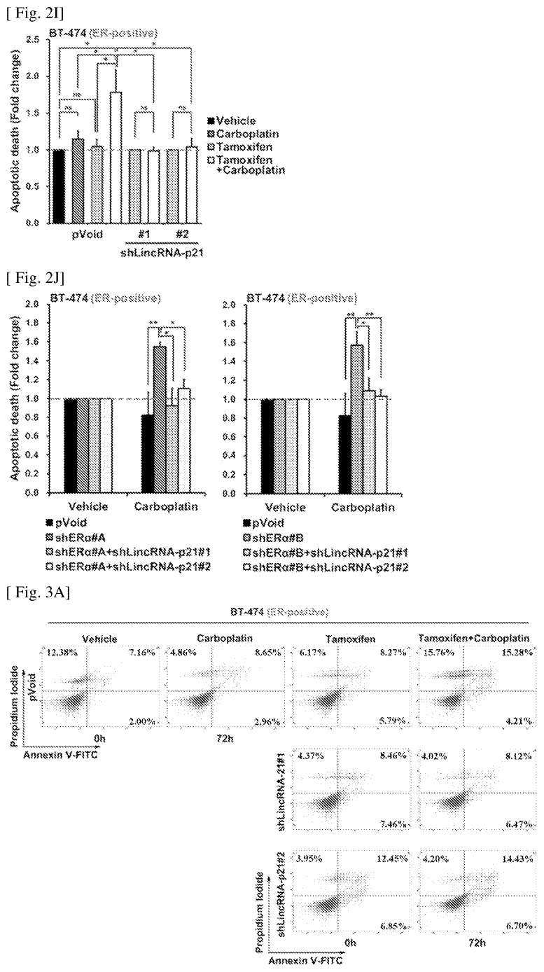

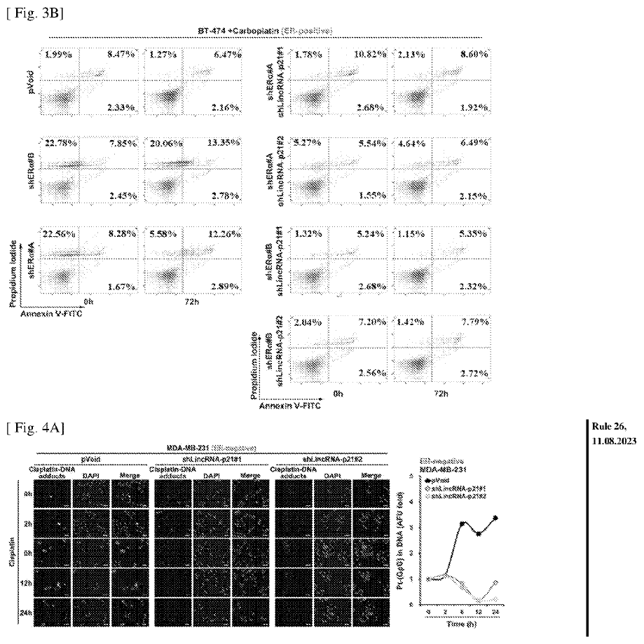

The roles of lincRNA-p21 in regulating DDB2-mediated DNA repair and chemoresistance remain unclear. The basal level of lincRNA-p21 was relatively higher in early stage (FIGS. 1A and 1B), smaller size (FIG. 1B), and ERα-negative (FIG. 1C) breast tumors. Induction of lincRNA-p21 by the tetracycline-inducible expression system in ERα-positive T47D breast cancer cells suppressed the tumor growth in a xenograft mice model (FIG. 2A), revealing its tumor suppressive role in breast cancer. In response to ex vivo treatments with carboplatin, the induction of lincRNA-p21 in primary human breast tumor tissues was declined accompanied with the advanced stages (FIG. 2B), tumor sizes (FIG. 2C), and the ERα-positive status (FIG. 2D). These clinical observations suggest a critical role for lincRNA-p21 in determining the chemosensitivity of breast cancer patients. Indeed, the levels of chemotherapy-induced lincRNA-p21 in various cell lines negatively correlated with their IC50 to the corresponding chemotherapeutic agents (FIG. 2E). Transient overexpression of lincRNA-p21 increased the carboplatin-induced apoptotic death of ERα-positive T-47D cancer cells (FIG. 2F). In contrast, silence of lincRNA-p21 expression in ERα-negative MDA-MB-231 cancer cells resulted in the reduction of cell apoptosis (FIG. 2G) and the cleavage of PARP and caspase3 (FIG. 2H) in response to carboplatin. Furthermore, silencing lincRNA-p21 also attenuated the sensitizing effect of tamoxifen (FIG. 2I and FIG. 3A) and ERα shRNA (FIG. 2J and FIG. 3B) on carboplatin-induced apoptotic death. Therefore, overexpression of lincRNA-p21 may overcome ERα-associated chemoresistance.

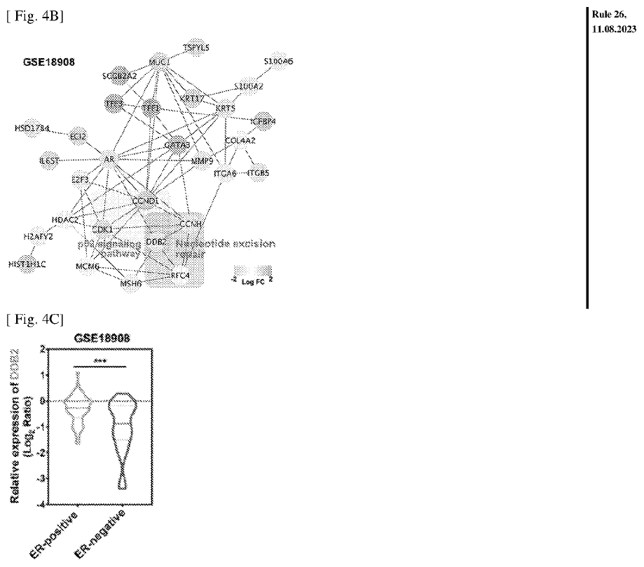

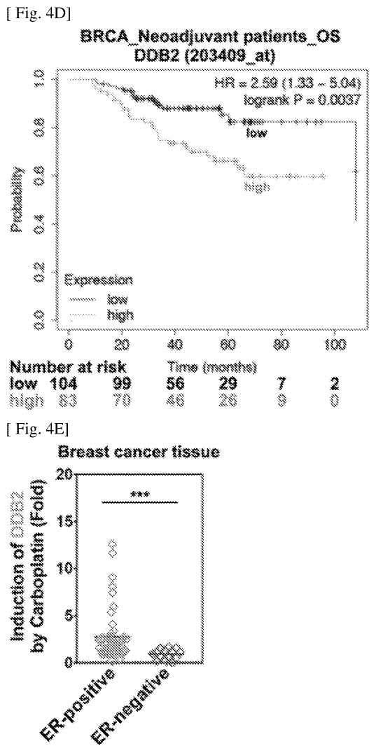

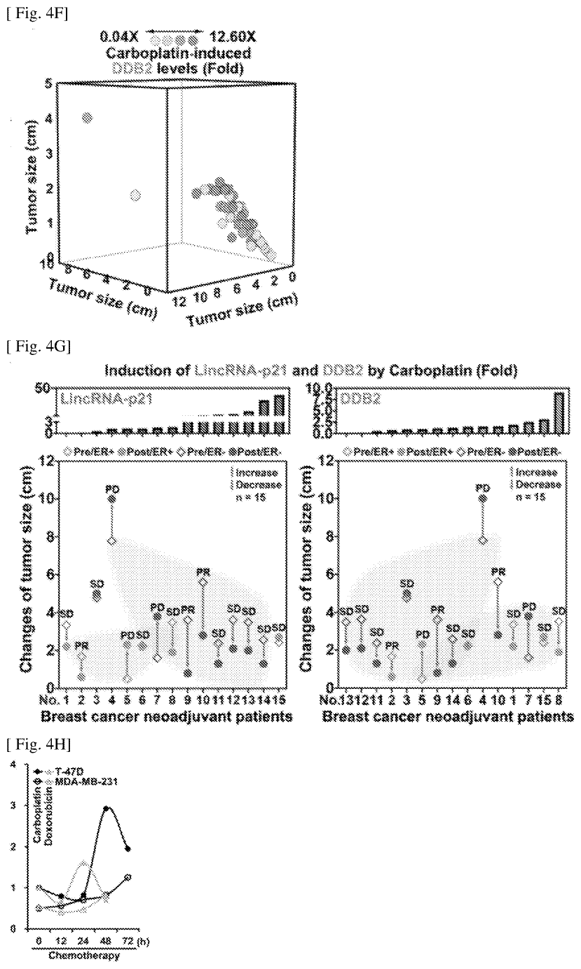

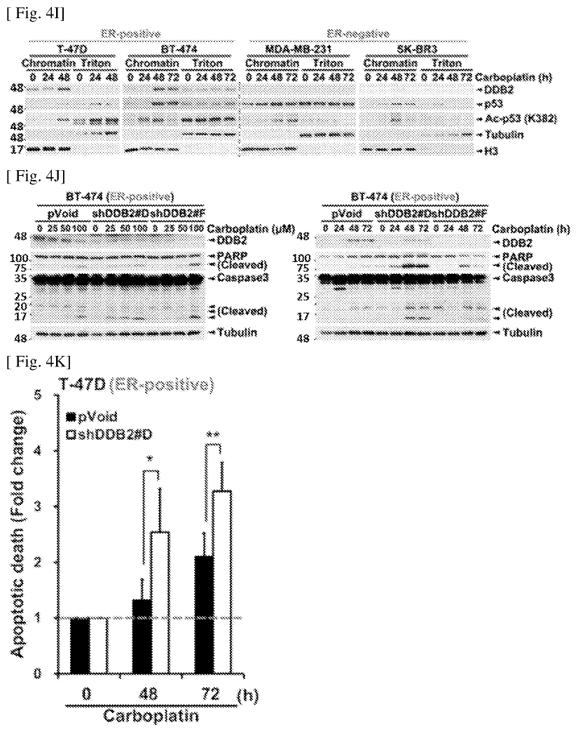

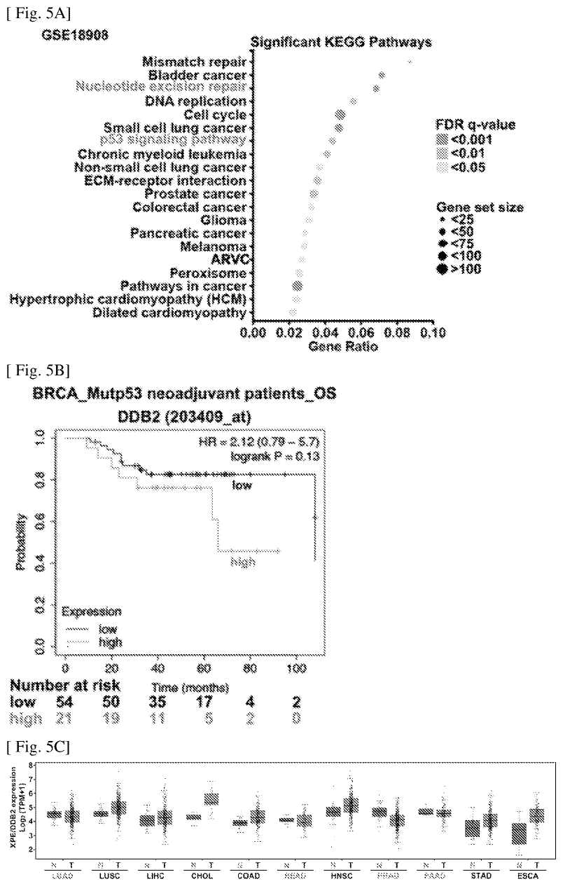

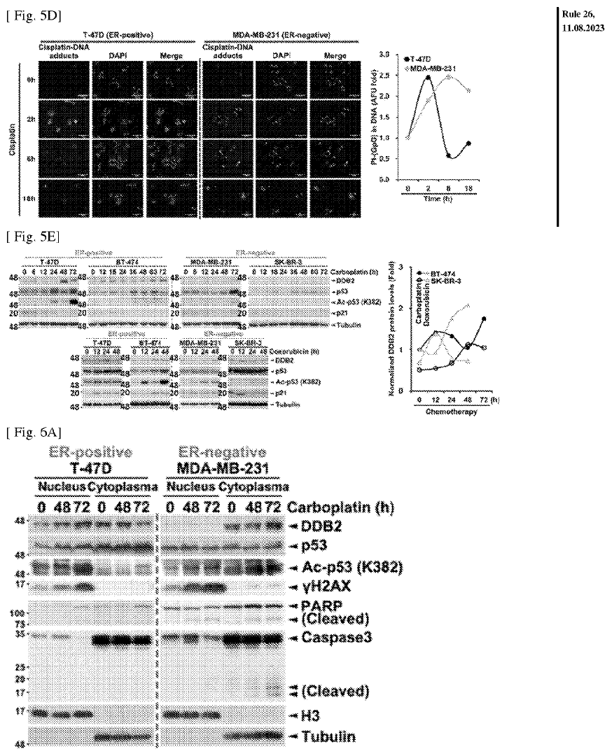

To further demonstrate the suppressive effect of lincRNA-p21 on DNA repair for chemosensitization, the cisplatin-DNA adducts were examined in immunocytological assays using an anti-cisplatin-modified DNA antibody. The results revealed that the induction of cisplatin-DNA adducts was suppressed by silencing lincRNA-p21 with shRNA in cisplatin-treated MDA-MB-231 cancer cells (FIG. 4A), indicating that lincRNA-p21 is able to increase chemosensitivity by suppressing DNA repair function. To determine the mechanisms by which lincRNA-p21 reduces ERα-mediated DNA repair, the GSE18908 dataset was employed to analyze differential gene expression profiles between human ERα-positive and -negative breast cancers. Among the ERα-associated pathways analyzed in the Kyoto Encyclopedia of Genes and Genomes (KEGG) pathway analysis (FIG. 5A), nuclear excision repair (NER) and p53 signaling, two critical pathways in the regulation of DNA repair and chemosensitivity, were upregulated in response to ERα expression. The STRING network further revealed that the expression of DDB2, a well-known p53-targeted downstream component of NER, was enriched in ERα-positive breast cancer (FIG. 4B) and may participate in the ERα/lincRNA-p21 axis-regulated chemosensitivity and DNA repair function. In addition, DDB2 levels were statistically higher in ER-positive breast cancer tissues (FIG. 4C). More importantly, the Kaplan-Meier Plot analysis revealed a correlation between higher DDB2 expression and poorer overall survival in all breast cancer patients (FIG. 4D) even with p53 mutation status (FIG. 5B) who received neoadjuvant chemotherapy. In addition, DDB2 is higher expressed in several different cancer types, such as lung cancer (LUSC), liver cancer (LIHC), cholangiocarcinoma (CHOL), colon cancer (COAD), head and neck squamous cell carcinoma (HNSC), stomach adenocarcinoma (STAD), and esophageal carcinoma (ESCA) (FIG. 5C). The ex vivo induction of DDB2 protein expression by carboplatin was also dramatically higher in ERα-positive than in ERα-negative tumor tissues (FIG. 4E) and correlated with tumor sizes (FIG. 4F). Furthermore, breast tumors from patients with a higher lincRNA-p21 but lower DDB2 inductions by ex vivo treatments with carboplatin correlated with their better clinical responses to adjuvant chemotherapies (FIG. 4G), suggesting that lincRNA-p21 may negatively regulate DDB2 expression to suppress NER for chemosensitization in breast cancer patients.

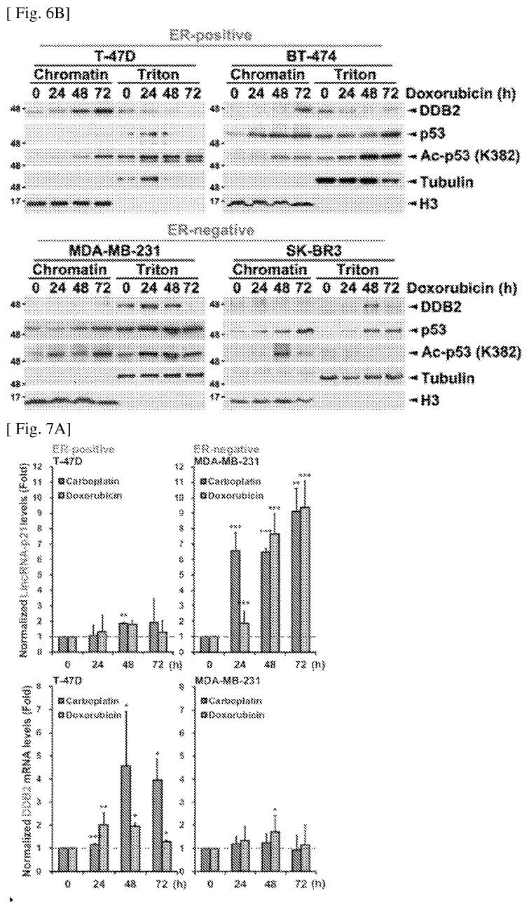

The present invention next investigated the involvement of DDB2-dependent NER in ERα-associated chemoresistance. Cisplatin-DNA adducts were induced by treatments with cisplatin within 2 hours in both ER-negative/chemosensitive MDA-MB-231 and ER-positive/chemoresistant T-47D cancer cells, and these DNA damages persisted for over 18 hours in MDA-MB-231 cancer cells but rapidly disappeared in T-47D cancer cells (FIG. 5D). Both carboplatin (FIGS. 4H and 5E) and doxorubicin (FIG. 5E) increased DDB2 expression in ERα-positive (T-47D and BT-474) but not ERα-negative (MDA-MB-231 and SK-BR-3) breast cancer cell lines. DDB2 recognizes and binds to damaged DNA sites in the nucleus as an essential initiator to further recruit other regulators involved in NER and is subsequently proteasomal degraded for the formation of the DNA repair complex. Hence, chemotherapy-induced nuclear translocation (FIG. 6A) and chromatin binding activity (FIGS. 4I and 6B) of DDB2 were also found in ERα-positive but not ERα-negative cancer cell lines. Furthermore, silencing DDB2 expression increased PARP or caspase3 cleavages in a dose- and time-dependent manner (FIG. 4J) and sensitized the ERα-positive cancer cells to carboplatin-induced apoptotic cell death (FIG. 4K). Taken together, these results suggest that DDB2 is a key NER initiator in mediating ERα-associated chemoresistance which can be targeted by lincRNA-p21.

### LincRNA-p21 Acts as a Scaffold of Cul-4 E3-Ligase Complex for DDB2 Proteasomal Degradation

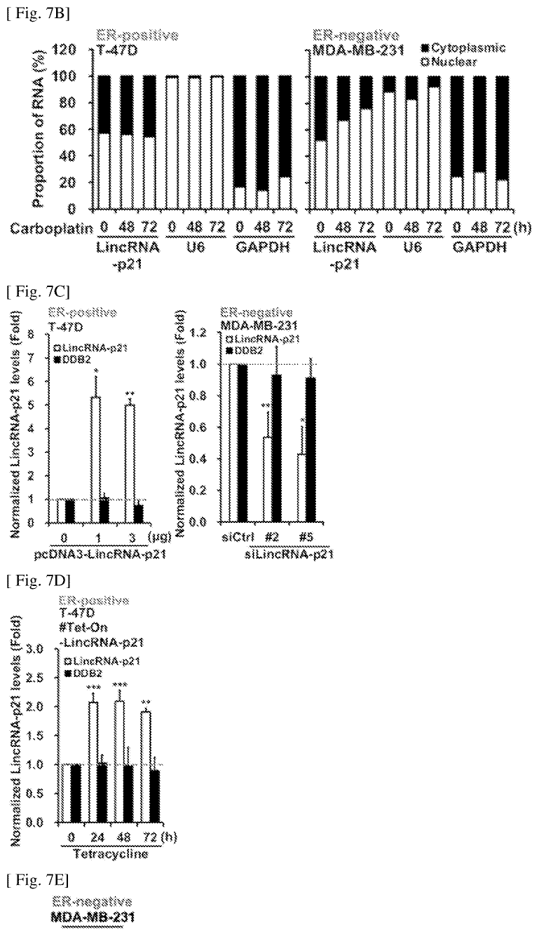

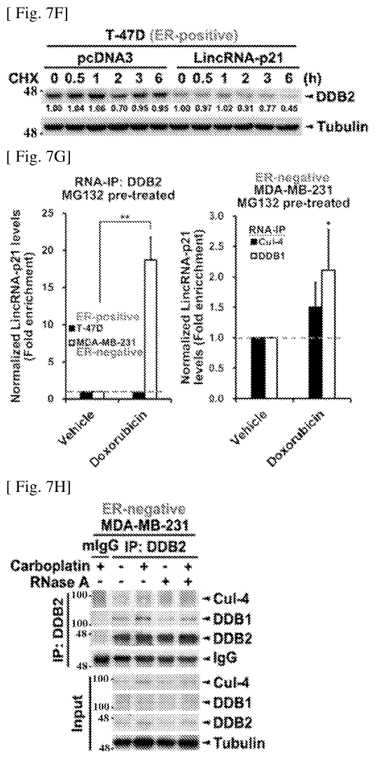

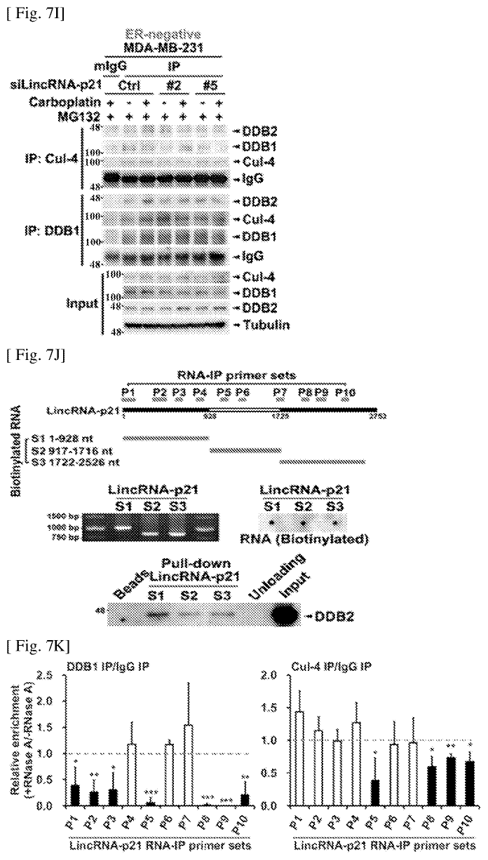

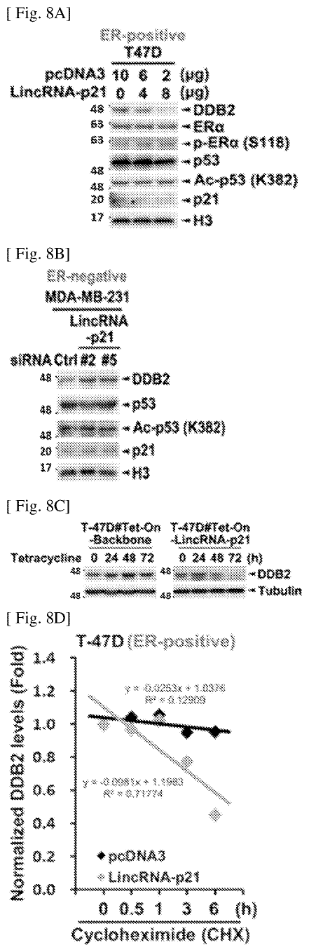

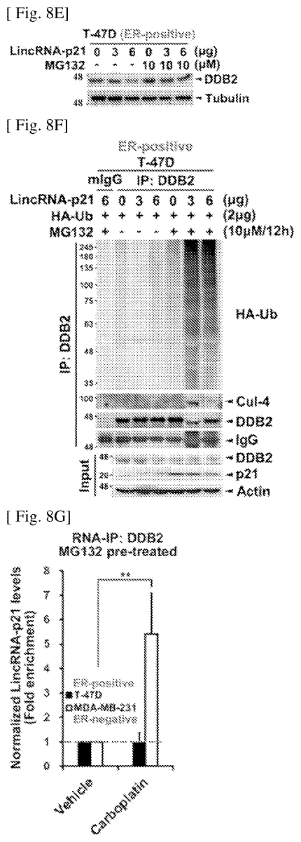

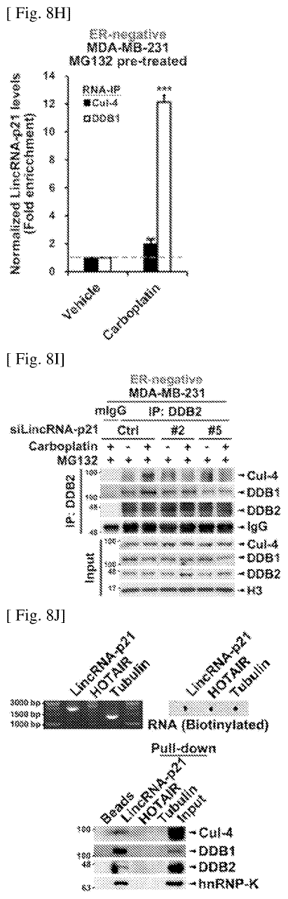

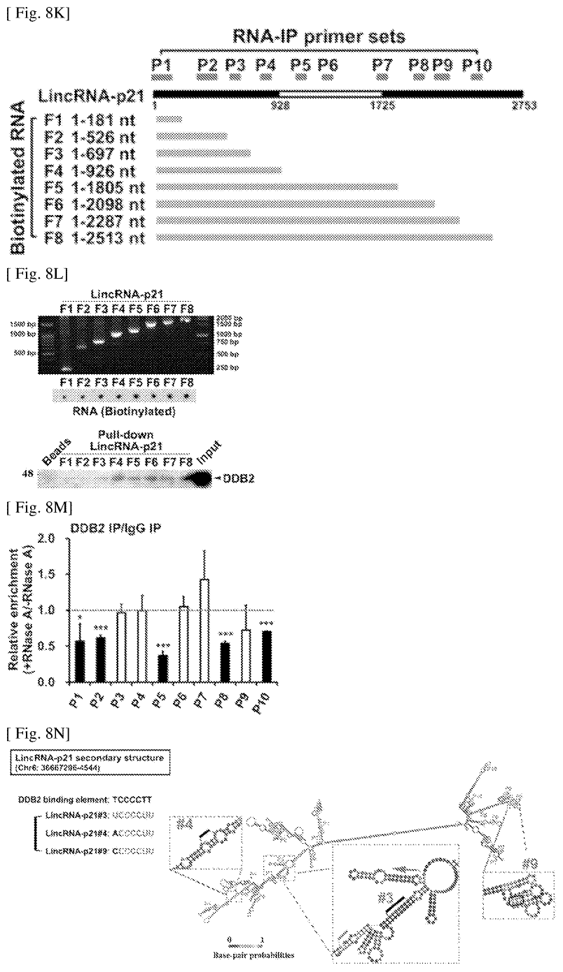

Treatments with carboplatin or doxorubicin time-dependently increased lincRNA-p21 expression in chemosensitive MDA-MB-231 cancer cells but not in chemoresistant T-47D cancer cells, and negatively correlated with the chemotherapy-induced DDB2 mRNA expression (FIG. 7A). Nuclear accumulation of lincRNA-p21 was also increased by carboplatin in MDA-MB-231 cancer cells but not in T-47D cancer cells (FIG. 7B) and inversely correlated with nuclear translocation of DDB2 (FIG. 6A). The present invention thus next investigated whether lincRNA-p21 regulates DDB2 expression and the underlying molecular mechanisms. Interestingly, DDB2 protein level was dose-dependently suppressed by lincRNA-p21 overexpression in T-47D cancer cells (FIG. 8A) and was enhanced by silencing lincRNA-p21 in MDA-MB-231 cancer cells (FIG. 8B) without affecting its mRNA level (FIG. 7C). Similarly, lincRNA-p21 induced by Tet-On control system also suppressed the DDB2 protein but not RNA levels (FIGS. 7D and 8C), implying the post-transcriptional downregulation of DDB2 by lincRNA-p21. Proteasome inhibitor MG132 enhanced DDB2 expression in lincRNA-p21-enriched MDA-MB-231 cancer cells (FIG. 7E). DDB2 protein stability was reduced by lincRNA-p21 overexpression in the presence of cycloheximide (CHX) (FIGS. 7F and 8D) and restored by MG132 (FIG. 8E), suggesting the involvement of lincRNA-p21 in regulating DDB2 proteasomal degradation. Overexpression of lincRNA-p21 enhanced the poly-ubiquitination of DDB2 in T-47D cancer cells (FIG. 8F). The role of lincRNA-p21 in regulating the complex formation of DDB2 with its E3-ligase Cul-4 and adaptor protein DDB1 was thereby addressed. The physical interaction between lincRNA-p21 and DDB2 in response to carboplatin (FIG. 8G) and doxorubicin (FIG. 7G) was observed in ERα-negative but not ERα-positive breast cancer cells in the RNA-IP assays. Carboplatin (FIG. 8H) and doxorubicin (FIG. 7G) also strongly increased the association of lincRNA-p21 with DDB1, but only moderately increased its association with Cul-4 in vivo. The interaction of DDB2 with DDB1 and Cul-4 complex was attenuated by RNase A treatment in vitro (FIG. 7H) and was disrupted by silencing lincRNA-p21 in vivo in the anti-DDB2 (FIG. 8I), anti-DDB1, and anti-Cul-4 (FIG. 7I) immunocomplexes. Next, the specific interactions of lincRNA-p21 with DDB2, DDB1, and Cul-4 in vitro were also validated in the RNA pull-down assays with biotinylated oligonucleotides (FIG. 8J). Together, these data indicate that lincRNA-p21 directly binds to DDB2/DDB1/Cul-4 and acts as the scaffold for the E3 complex formation.

To examine the specific and essential regions of lincRNA-p21 for binding to DDB2, the present invention next synthesized different segments of lincRNA-p21 (S1: Exon1, S2: Intron, S3: Exon2) (FIG. 7J) or deletion fragments (F1-F8) (FIG. 8K) to analyze their binding activity to DDB2. In the in vitro RNA pull-down assays, S1 and F3-F8 presented stronger binding efficacy on DDB2, suggesting that the region 526-926 was required the DDB2-binding activity. In the RNA-IP analysis, the pulled-down RNA in anti-DDB2 immunoprecipitates were digested with or without RNase A in vitro followed by RT-qPCR with various primer sets for the amplification of different regions (P1-P10) as illustrated in FIG. 8K. The regions of lincRNA-p21 at P3, P4, P6, P7, and P9 were resistant to the RNase A digestion probably due to the protection by binding to DDB2, further revealing the potential DDB2-binding regions in vivo (FIG. 8M). Interestingly, the binding regions of lincRNA-p21 for DDB1 and Cul-4 were similar to that for DDB2 (FIG. 7K). DDB2 has been reported as a transcription factor with the binding affinity to the specific consensus elements on the promoters of its target genes. It is noteworthy that this consensus sequence can be found within the regions P3, P4, and P9 of lincRNA-p21 and fold as secondary structures (P3, P4, and P9), implying that these three elements as the potential binding sites for DDB2 (FIG. 8N). These results suggest that two regions (527 to 926 and 2099 to 2287) of lincRNA-p21 containing elements #3, #4, and #9 are required for the interaction with DDB2.

Potential Short lincRNA-p21 Elements Act as DDB2 Inhibitors for Chemosensitization

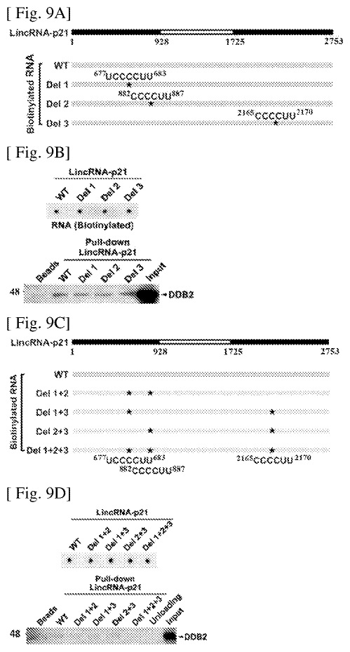

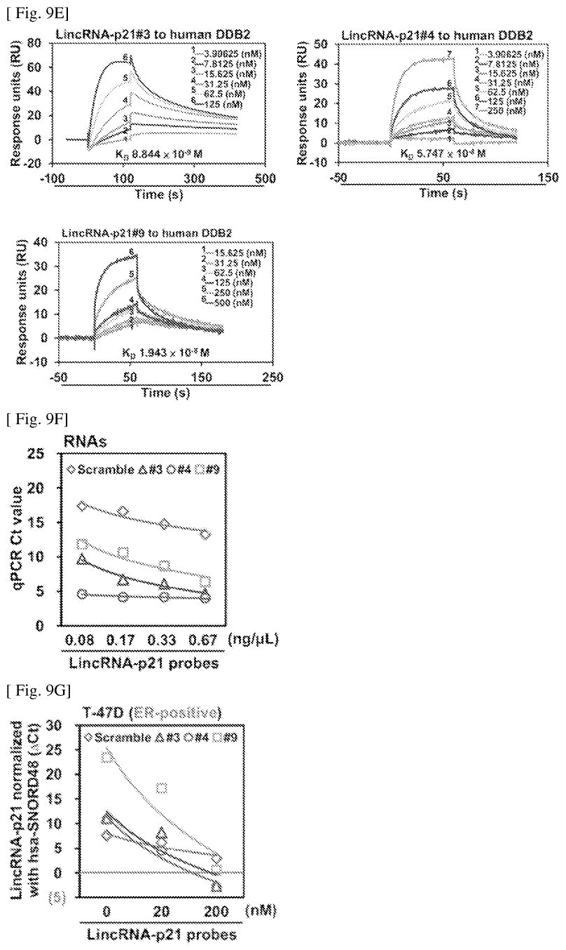

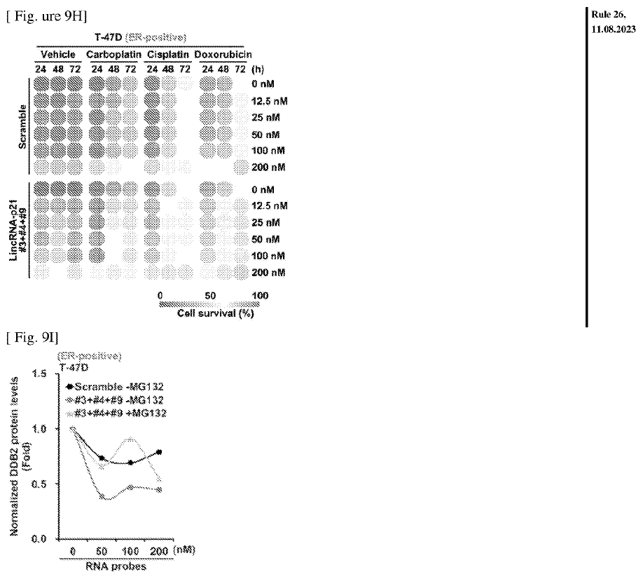

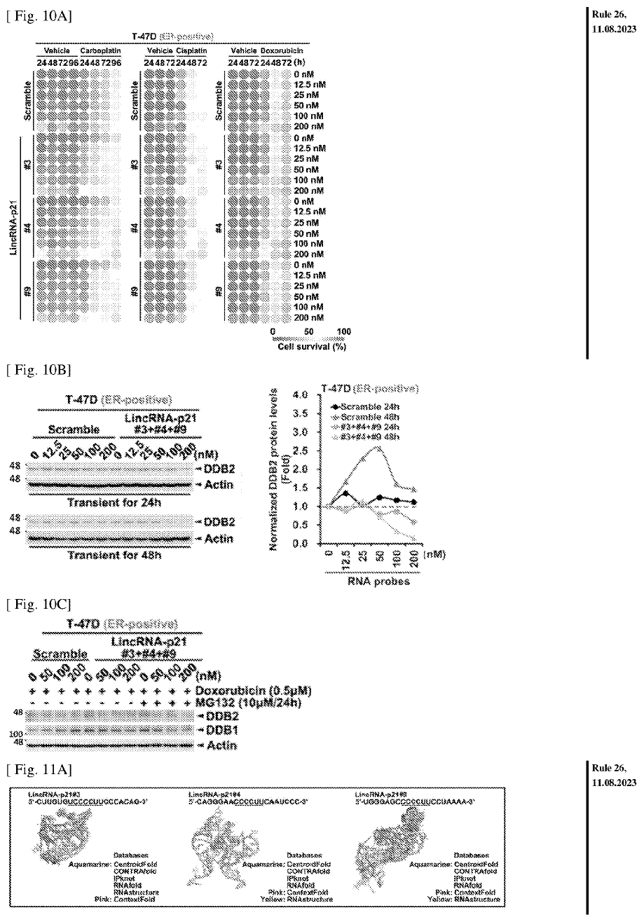

To demonstrate that necessity of the putative elements (P3, P4, and P9) of lincRNA-p21 for the interaction with DDB2 in vitro, these essential elements marked by a star symbol in FIGS. 9A and 9C were deleted. The protein level of DDB2 pulled down by the biotinylated lincRNA-p21 full-length probes was slightly attenuated by individual deletion at P3, P4, or P9 (Del 1, Del 2, or Del3) (FIG. 9B) and was almost abolished by the combinatorial mutations at all three elements (Del 1+2+3) (FIG. 9D). In addition, the synthesized RNA oligonucleotides corresponding to these DDB2-binding elements (#3, #4, and #9) of lincRNA-p21 showed strong binding activities to recombinant DDB2 protein in a dose-dependent manner with K_{D} values of 10⁻⁹ to 10⁻⁸ M in SPR analysis (FIG. 9E). The synthesized short lincRNA-p21 elements showed comparable quantities with scramble control in a dose-dependent manner in vitro (FIG. 9F). Next, the present invention transiently transfected the short lincRNA-p21 elements into T-47D cancer cells and detected the delivery efficacy by qRT-PCR analysis (FIG. 9G). Compared to the scramble, mixed three short lincRNA-p21 elements (#3+ #4+ #9) (Linc-p21s) enhanced the chemosensitivity of the ER-positive/chemoresistant T-47D cancer cells in response to platinum drugs and doxorubicin in a dose-dependent manner (FIG. 9H) although single short lincRNA-p21 element (#3, #4, or #9 alone) only showed minor chemosensitizing effects (FIG. 10A). In addition, the DDB2-targeting effect of Linc-p21s was evidenced by the downregulations of DDB2 protein expression after 24 hours of treatments (FIG. 10B), which were prevented by pre-treatments with MG132 (FIGS. 91 and 10C).

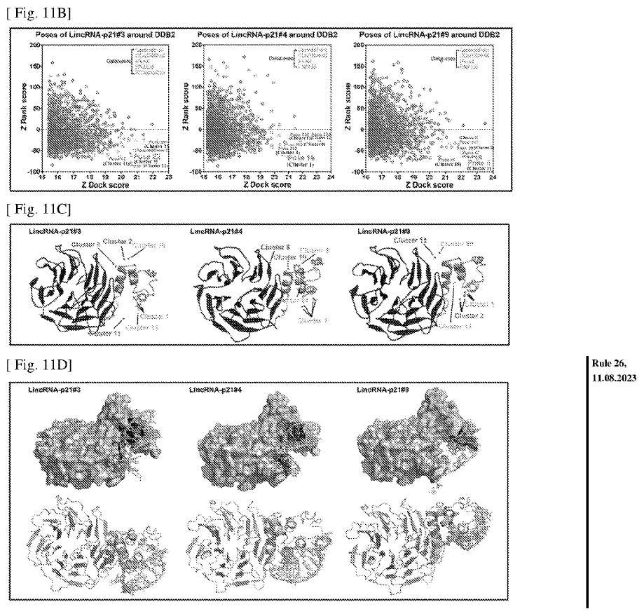

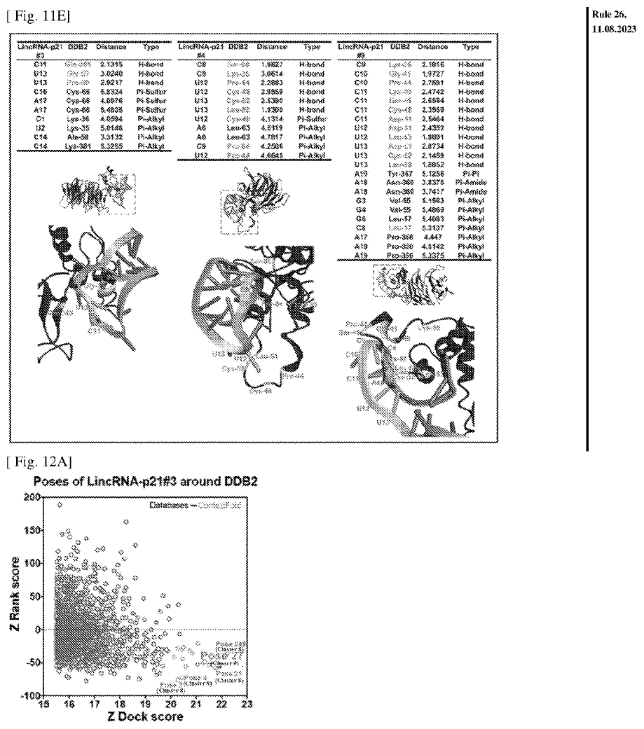

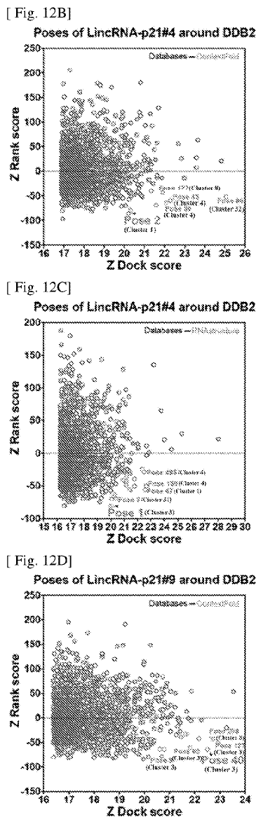

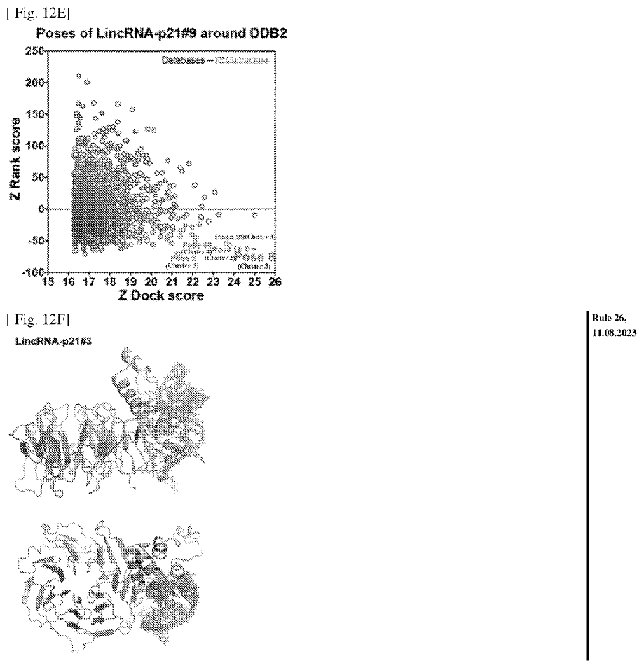

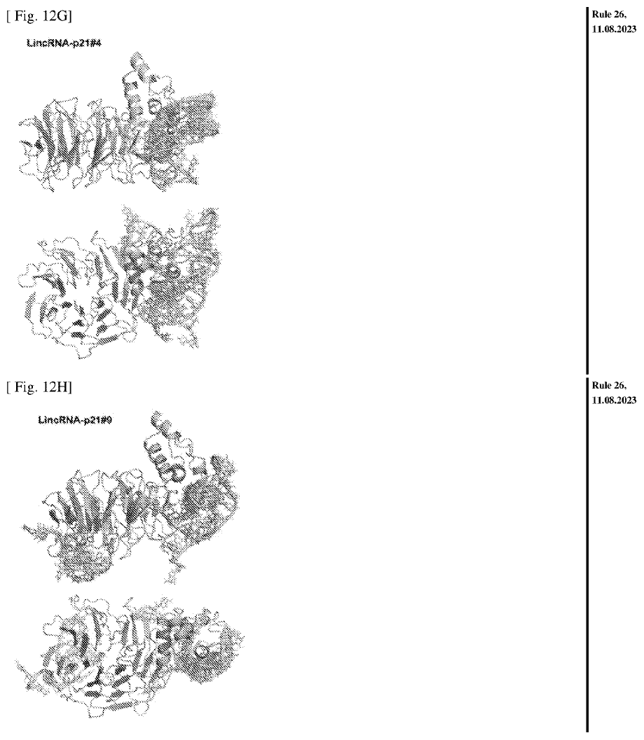

To further explore the potential conformation between these three short lincRNA-p21 elements and DDB2 in silico, RNAComposer which includes six databases, CentroidFold, CONTRAfold, IPknot, RNAfold, RNAstructure, and ContextFold was used to predict the 3D structure of short lincRNA-p21 elements (Linc-p21s). Interestingly, the same structural conformation predicted by at least four databases (CentroidFold, CONTRAfold, IPknot, and RNAfold) (FIG. 11A) was obtained and utilized for further molecular docking analysis with the DDB2 protein (PDB: 4E54). Macromolecular docking was calculated by using the ZDOCK docking program and the potential DDB2-binding poses of short lincRNAs from different predictive databases with a lower Z Rank score and higher Z Dock score were considered (FIGS. 11B and 12A-12E). These potential poses were then classified into different clusters (FIG. 11C) and presented in a similar binding region but with different interacting models. Furthermore, the most potential pose was selected from the largest cluster with the best Z Rank as well as Z Dock score to represent the interaction between short lincRNA-p21 elements and DDB2 (FIGS. 11D and 12F-12H). The interaction sites and distances between short lincRNA-p21 elements and DDB2 were also calculated (FIG. 11E). In the 3D structures, the central nucleotides of the short lincRNA-p21 elements (C8, C9, C10, C11, U12, and U13) interacted with the most potentially involved amino acids (Lys-35, Pro-44, Cys-48, Cys-52, and Leu-53) or other amino acids of DDB2 by hydrogen bonding and Pi-Alkyl interaction respectively (FIG. 11E). In the results of the complex structure, all three short lincRNA-p21 elements were coiled with the α-helix of DDB2 N-terminus, which is important and responsible for the interaction with DDB1 to adapt the E3-ligase Cul-4 (FIG. 11D). It was reasonable that the short lincRNA-p21 coiled with DDB2 to stabilize the formation of the DDB2/DDB1/Cul-4 E3 ligase complex and to enhance the poly-ubiquitination and degradation of DDB2.

Short lincRNAp21s Packaged with Chemotherapy in Exosome Showed Promising Chemosensitizing Effects

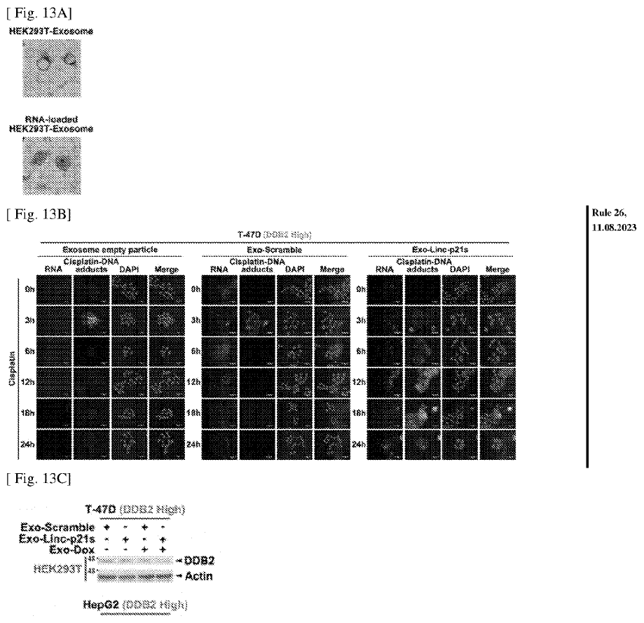

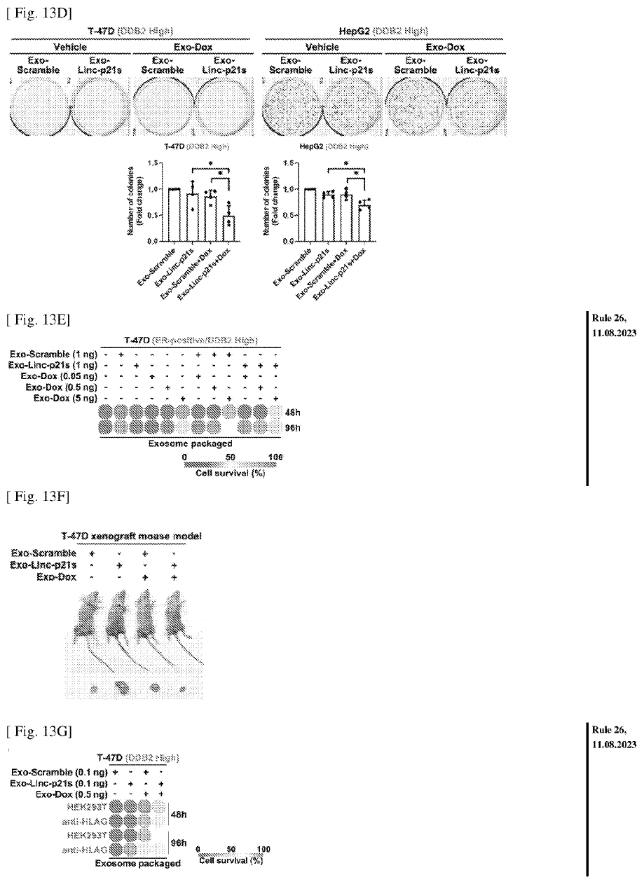

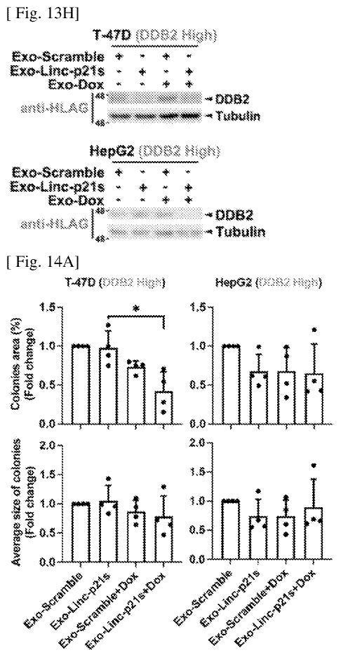

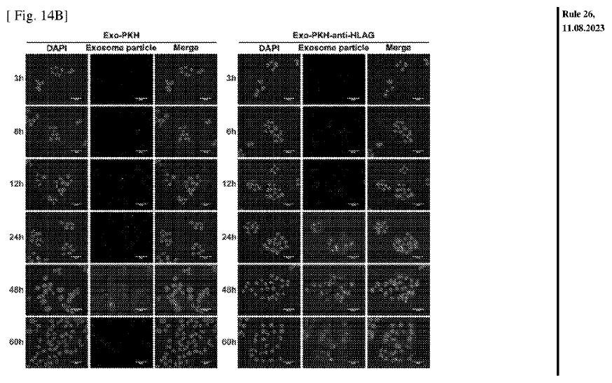

The delivery efficacy, tumor-targeting specificity, and stability of Linc-p21s in vivo are crucial for the development of an RNA-based therapeutic strategy for cancer patients. To increase the delivery efficacy of Linc-p21s in the human body, exosomes were employed as the delivery system for the treatment strategy. Transmission electron microscope (TEM) analysis showed no difference in the size and shape between empty and lincRNAp21-packaged exosome (FIG. 13A). To validate the function of exosome-packed Linc-p21s (exoLinc-p21s) in DNA repair, the cisplatin-DNA-adducts assay was performed and demonstrated that the exoLinc-p21s prolonged the existence of cisplatin-DNA-adducts from 3 hours to 24 hours, suggesting that exoLinc-p21s was able to increase chemosensitivity by reducing DNA repair (FIG. 13B). In the exosome packed with or without doxorubicin (exoDox), the ability of exoLinc-p21s to inhibit doxorubicin-induced DDB2 expression was confirmed by western blot analysis (FIG. 13C). Furthermore, exoLinc-p21s reduced the growth of T-47D breast cancer cells and HepG2 (high DDB2-expressing) hepatocarcinoma cells in colony formation assays (FIGS. 13D and 14A) and, similar to the results from the transient transfection system, exoLinc-p21s containing Ing of short Linc-p21s synergized the cytotoxicity of doxorubicin (FIG. 13E). In the xenograft mouse model, the Exo-Linc-p21s also enhanced the anti-tumor activity of doxorubicin in vivo (FIG. 13F). To enhance the tumor-targeting specificity of exoLinc-p21s, antibody against HLAG, which is highly expressed in most tumors, was engineered onto the surface of exoLinc-p21s to increase tumor-specificity. Indeed, the exoLinc-p21s with anti-HLAG had an earlier and more prolonged accumulation in the uptake efficiency of the T-47D cancer cells (FIG. 14B). Importantly, the anti-HLAG engineered exoLinc-p21s exhibited a better cytotoxic effect than exoLinc-p21s without anti-HLAG (FIG. 13G) and a stronger suppression of DDB2 protein expression (FIG. 13H).

Taken together, the data of the present invention demonstrated that Linc-p21s packed with chemotherapy in exosome (exoLinc-p21s) effectively targeted DDB2 protein to suppress DNA repair for chemosensitization, which could be a potential and novel RNA-based DDB2 inhibitor to enhance chemosensitivity in patients with various DDB2-expressing tumors.

Those skilled in the art recognize the foregoing outline as a description of the method for communicating hosted application information. The skilled artisan will recognize that these are illustrative only and that many equivalents are possible.

1. A composition comprising a sequence of long intergenic non-coding RNA-p21 (lincRNA-p21), wherein the sequence of lincRNA-p21 is selected from the group consisting of SEQ ID NO: 1, SEQ ID NO: 2, and SEQ ID NO: 3.

2. A use of a composition for preparing a drug for treating cancer, wherein the composition comprises a sequence of long intergenic non-coding RNA-p21 (lincRNA-p21) and a chemotherapeutic agent, wherein the sequence of lincRNA-p21 is selected from the group consisting of SEQ ID NO: 1, SEQ ID NO: 2, and SEQ ID NO: 3.

3. The use of claim 2, wherein the sequence of lincRNA-p21 enhances the sensitivity of the cancer to the chemotherapeutic agent by inhibiting the expression of DDB2.

4. The use of claim 2, wherein the cancer comprises the cancer with the high expression of DDB2.

5. The use of claim 2, wherein the cancer has poor response or drug-resistance for the chemotherapeutic agent.

6. The use of claim 2, wherein the cancer comprises breast cancer and liver cancer.

7. The use of claim 6, wherein the cancer cells of the breast cancer have mutant p53.

8. The use of claim 7, wherein the cancer cells of the breast cancer are estrogen receptor-positive and have mutant p53.

9. The use of claim 2, wherein the chemotherapeutic agent comprises carboplatin, cisplatin or doxorubicin.

10. The use of claim 2, wherein the composition further comprises a pharmaceutically acceptable carrier.

11. The use of claim 10, wherein the pharmaceutically acceptable carrier comprises a liposome, a nanoparticle, an exosome, a micelle, a polymeric matrix or a gel matrix.

12. The use of claim 11, wherein the sequence of lincRNA-p21 is contained in the exosome.

13. The use of claim 2, wherein the composition further comprises a target molecule for binding to a biomarker on cancer cells.

14. The use of claim 13, wherein the target molecule comprises an anti-HLAG antibody.


SEQUENCE LISTING (3 sequences)

SEQ ID NO: 1 - RNA, 20 nt, synthetic construct
  source 1..20: mol_type = other RNA
cttgtgtccccttcccacag

SEQ ID NO: 2 - RNA, 20 nt, synthetic construct
  source 1..20: mol_type = other RNA
cagggaaccccttcaatccc

SEQ ID NO: 3 - RNA, 20 nt, synthetic construct
  source 1..20: mol_type = other RNA
tgggagcccccttcctaaaa